SEQUENCE LISTING (8 sequences)

SEQ ID NO: 1 - protein, 9 aa, Artificial Sequence
  Description of Artificial Sequence Synthetic peptide
FQPQNGQFI

SEQ ID NO: 2 - protein, 9 aa, Artificial Sequence
  Description of Artificial Sequence Synthetic peptide
WPIACRSTI

SEQ ID NO: 3 - protein, 9 aa, Artificial Sequence
  Description of Artificial Sequence Synthetic peptide
KAVYNFATC

SEQ ID NO: 4 - protein, 11 aa, Artificial Sequence
  Description of Artificial Sequence Synthetic peptide
SGVENPGGYCL

SEQ ID NO: 5 - protein, 9 aa, Artificial Sequence
  Description of Artificial Sequence Synthetic peptide
GYCLTKWMI

SEQ ID NO: 6 - protein, 9 aa, Artificial Sequence
  Description of Artificial Sequence Synthetic peptide
LYLVCGERG

SEQ ID NO: 7 - protein, 9 aa, Artificial Sequence
  Description of Artificial Sequence Synthetic peptide
KYNKANAFL

SEQ ID NO: 8 - protein, 15 aa, Artificial Sequence
  Description of Artificial Sequence Synthetic peptide
SHLVEALYLVCGERG

(19) United States
(12) Patent Application Publication
(10) Pub. No.: US 2007/0190045 A1
(43) Pub. Date: Aug. 16, 2007

(54) Anti-CD3 and antigen-specific immunotherapy to treat autoimmunity

(76) Inventors: Kevan Herold, Scarsdale, NY (US); Matthias Von Herrath, Del Mar, CA (US); Jeffrey A. Bluestone, San Francisco, CA (US)

Correspondence Address: WilmerHale/Columbia University, 399 PARK AVENUE, NEW YORK, NY, 10022, US

(21) Appl. No.: 11/498,381

(22) Filed: Aug. 3, 2006

Related U.S. Application Data
(63) Continuation of application No. PCT/US05/03712, filed on Feb. 4, 2005.
(60) Provisional application No. 60/541,959, filed on Feb. 4, 2004.

(51) Int. Cl. A61K 39/395; A61K 38/20; A61K 39/00

(52) U.S. Cl. 424/131.1; 424/184.1; 424/85.2; 424/144.1

(57) ABSTRACT

The invention provides methods for treating autoimmunity and for reestablishing tolerance. The methods involve the coadministration of anti-CD3 antibodies and self-antigens. The coadministration has the potential to provide a synergistic effect of protecting or reducing autoaggressive immune processes and/or of reestablishing tolerance towards self-antigens. An underlying rationale behind the methods is that the administration of self-antigens together with anti-CD3 antibodies can alter the response to those self-antigens and prevent progression of autoimmunity. By rechallenging with the autoantigens and stimulating the non-pathogenic response, the blockade of the autoimmune process can be maintained. Preclinical evidence provided herein shows that the combination of anti-CD3 and autoantigen is synergistic in reversing autoimmune diabetes, and therefore, suggests that combination therapy of anti-CD3 and self-antigen may provide synergistic protection in reversing other autoimmune disorders.

This application claims the benefit of and is a continuation to International Application No. PCT/US2005/003712, filed Feb. 4, 2005, which claims priority to U.S. Provisional Patent Application Ser. No. 60/541,959, filed Feb. 4, 2004, both of which are hereby incorporated in their entirety.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

The invention disclosed herein was made with U.S. Government support from National Institutes of Heath Grant 1 R21 DK069872-01. Accordingly, the U.S. Government has certain rights in this invention.

### BACKGROUND OF THE INVENTION

The immune systems of healthy individuals are tolerant to the body's own self-antigens. Tolerance is a state of immunological unresponsiveness to an antigen, and autoimmunity occurs when tolerance is not present for a self-antigen. Therapies for autoimmune diseases have been hampered due to the fact that the cause of autoimmunity is often multifactorial with complicated etiologies involving multiple autoantigens as targets.

Due to the complicated nature of autoimmunity, prior therapies attempt to achieve a general suppression of the immune system. Most immune suppressive agents prevent T cell responses by depletion or inactivation of T cells. For example, glucocorticoids and the calcineurin inhibitors, such as cyclosporine A and FK-506, block cytokine gene transcription, preventing the production of T cell growth factors. Other agents, such as Campath 1H, cause prolonged depletion of T cells. While these approaches are effective in the short term, their effects are not antigen specific and may not persist after the drugs are discontinued. Hence, true immunologic tolerance, in which an immune response does not occur after an immune suppressive agent is withdrawn, is rarely achieved.

In contrast, the monoclonal antibody (mAb) against the CD3 molecule has induced tolerance to autoimmunity in murine models of type 1 diabetes mellitus. Treatment with anti-CD3 mAb reversed diabetes in the NOD mouse and prevented recurrent immune responses toward transplanted syngeneic islets. This was achieved without the need for continuous immune suppression and persisted at a time when T cell numbers were not depleted and were quantitatively normal. Another approach is to induce specific immunological unresponsiveness by administering self-antigens. However, administering anti-CD3 or self-antigen alone may be limited in their duration of tolerance induction or in their efficacy after disease presentation.

### SUMMARY OF THE INVENTION

The present invention provides methods that involve the use of both anti-CD3 antibodies (or other CD3 ligands) and self-antigens in order to treat autoimmunity. The administration of both anti-CD3 antibodies and self-antigens (i.e., “coadministration” as used herein) has the potential to provide a synergistic effect for treating autoimmune disorders over the administration of either anti-CD3 antibodies or self-antigens alone, where the synergistic effect can be manifested by an increased immunological tolerance to self-antigens that are the target of autoimmune responses. Not only is a synergistic effect that might be provided by coadministration unexpected and/or uncertain, but the possibility that coadministration itself can help treat autoimmunity is also unexpected because is reasonable to believe that (1) anti-CD3 treatment may result in a general immunosuppressive effect that overrides or cancels-out any tolerance induction generated by the self-antigens, or (2) either anti-CD3 or self-antigen administration may result in the aggravation of existing autoaggressive phenomena. Without being bound by theory, the invention provides that anti-CD3 ‘resets’ the immune system thereby opening a window for some therapeutic interventions with antigen specific treatments to induce regulation that can maintain long-term tolerance.

In one aspect, the invention provides a method for restoring or inducing tolerance to a self-antigen in a subject, the method comprising administering to the subject: (a) an anti-CD3 antibody, and (b) the self-antigen; wherein the anti-CD3 antibody and the self-antigen are administered in an amount sufficient to restore or induce tolerance to the self-antigen in the subject.

In one aspect, the invention provides a method for reducing, inhibiting or preventing an immune response against a self-antigen in a subject, the method comprising, administering to the subject: (a) an anti-CD3 antibody, and (b) the self-antigen, wherein the anti-CD3 antibody and the self-antigen are administered in an amount sufficient to reduce, inhibit or prevent an immune response in the subject against the self-antigen. The immune response can be a humoral or cell-mediated immune response.

In one aspect, the invention provides a method for producing (or generating or inducing) antigen specific T regulatory cells in a subject, the method comprising administering to the subject: (a) an anti-CD3 antibody, and (b) a self-antigen, wherein the anti-CD3 antibody and the self-antigen are administered to the subject in an amount sufficient for the production in the subject of T regulatory cells. In one aspect, the T regulatory cells can comprise a T cell receptor (TCR) specific for the self-antigen or other autoantigens.

In one aspect, the invention provides a method for restoring or establishing (or inducing) tolerance to a self-antigen, the method comprising: (a) administering to a subject: (i) an anti-CD3 antibody, and (ii) the self-antigen; (b) isolating T regulatory cells (and in another aspect, the T regulatory cells that are isolated comprise a T cell receptor (TCR) specific to the self-antigen); (c) incubating the T regulatory cells in vitro under growth conditions (or expanding the number of the T regulatory cell population that was isolated); and (d) administering to the subject the T regulatory cells from step (c) so as to restore or establish tolerance to the self-antigen. Step (c) can comprise, for example, incubating the T regulatory cell population with IL-2. Step (c) can further comprise incubating the T regulatory cells with the anti-CD3 antibody and the self-antigen. Additionally or alternatively, step (c) can further comprise incubating the T regulatory cells with antigen presenting cells (APCs) and the self-antigen. The APCs can be obtained from the subject, or the APCs can be obtained from other subjects that are syngeneic to the subject of the method. The T regulatory cells can be isolated from blood or lymph samples, for example, from the subject.

In the aspects of the invention that relate to isolating and/or inducing T regulatory cells, T regulatory cells can express on their cell surface, for example, CD4 and CD25; CD4, CD25 and CD62L; CD25, CD45RO, CD62L and GITR; CD25, FoxP3, GITR, CTLA4, CD62L and CD45RO; CD4, CCR4, CD62L and CD45RO; or subsets of CD8 T cells that express CD25. The isolation of T regulatory cells can be conducted, for example, by flow cytometry or magnetic bead methods that are able to separate populations based upon their cell surface expression or of certain proteins produced inside the cell, for example IL-4, IL-10 or TGF-beta. Flow cytometry is also able to identify intracellular protein expression, and therefore, the methods are not limited to the extracellular presentation of proteins on cell surfaces.

The methods of the invention are generally directed towards the treatment of autoimmune diseases and disorders. For example, subjects of the present methods can be suffering from Graves disease, Hashimoto's thyroiditis, hypoglyceimia, multiple sclerosis, mixed essential cryoglobulinemia, systemic lupus erthematosus, Type I diabetes, or any combination thereof. In one aspect, the subjects of the present methods suffer from autoimmune responses that involve T-cells or B-cells that have an antigenic specificity, or T-cell receptor (TCR) or B-cell receptor (BCR) specificity, for a self-antigen.

In the invention, the administration of self-antigen can involve a self-antigen that comprises a protein or a peptide fragment of the protein. For example, the self-antigen can comprise a thyroid-stimulating hormone receptor, thryoglobulin, throid peroxidase, myelin basic protein, glutamic acid decarboxylase (GAD65), islet cell antigen-2 (IA-2), insulin, proinsulin, or heat shock protein 60 (HSP 60), or any combination thereof, including fragments or mutants thereof.

In one aspect, the invention provides a method for treating Type I diabetes (T1D), the method comprising administering to a subject: (a) an anti-CD3 antibody, and (b) a self-antigen, wherein the anti-CD3 antibody and the self-antigen are administered in an amount sufficient to treat the underlying cause of Type I diabetes, which is an immunologically mediated destruction of the insulin producing cells in the Islets of Langerhans. The destruction of the insulin producing cells results in insufficient insulin production to meet metabolic demands causing elevated glucose levels, and if severe, ketosis. In another aspect, the anti-CD3 antibody and the self-antigen are administered in an amount sufficient to treat Type I diabetes or to treat one or more symptoms associated with Type I diabetes. Symptoms associated with Type I diabetes include, but are not limited to, reduced insulin production, abnormal blood glucose levels, destruction of insulin producing cells, and abnormal C peptide levels. The self-antigen can comprise, for example, an islet cell antigen. Specific self-antigens include, but are not limited to, insulin, proinsulin, proinsulin II, insulin B9-23 peptide, a proinsulin peptide without a cytotoxic T-lymphocyte epitope, insulin C13-A5 peptide, glutamic acid decarboxylase (GAD65), islet cell antigen 512/IA-2, islet cell antigen p69, and heat shock protein 60 (HSP 60).

In the methods, the anti-CD3 antibody and the self-antigen can be initially administered on the same day. By coadministration, anti-CD3 and antigens are coadministered when their dosing regimens overlap. In one aspect, coadministration dosing regimens are designed to ensure that the anti-CD3 antibodies and the antigens are encountered at least at one time point essentially simultaneously by the subject's immune system. Thus, for example, on day 1 of treatment, both anti-CD3 and antigen can be administered, and following day 1, anti-CD3 and antigen can be administered on different days. Coadministration is important because the invention has the potential to provide a synergistic protective effect (i.e., reestablishing/inducing tolerance, reducing autoaggressive responses, or generally reducing pathogenic effects of autoimmunity) that can be the result of the administration of both anti-CD3 antibody and the self-antigen. In other words, coadministration can provide the scenario where anti-CD3 administration has an effect on self-antigen administration, or vice versa. Anti-CD3 and self-antigen do not therefore need to be administered at the same time. However, they do need to administered close enough in time such that their effects upon the immune response can synergize.

In one aspect of the methods, the anti-CD3 antibody is a monoclonal antibody The antibody, for example, can comprise an IgG molecule. The antibody can be humanized (i.e, a chimera of rodent and human amino acid sequences) or fully human. In one aspect, the antibody should at least be bivalent (i.e., have at least two-antigen binding sites that have the same specificities). The anti-CD3 antibody can comprise an antibody subsequence or fragment. The antibody fragment can comprise, for example, a (Fab′)₂ molecule. In one aspect, the antibody fragment cannot be specifically bound by an Fc Receptor (i.e, the Fc-receptor binding portion of the immunoglobulin is either mutated or deleted). In one aspect, the anti-CD3 antibody comprises a non-mitogenic antibody. In one aspect, the anti-CD3 antibody comprises an OKT3 antibody. The OKT3 antibody can be a variant or mutant of the original OKT3 antibody, for example, a human (or humanized) OKT3γ (Ala-Ala) antibody.

In the present methods, the administration of self-antigen can comprise administration of an expression vector that encodes for the self-antigen, such that the expression vector produces the self-antigen in vivo.

In the present methods, the anti-CD3 antibody should be administered intravenously. Also, the self-antigen can be administered intranasally, orally, subcutaneously, intramuscularly, or intravenously. The anti-CD3 antibody and the self-antigen can be administered in/with a pharmaceutically acceptable carrier, excipient or diluent.

Thus, the invention involves the use of anti-CD3 antibodies and self-antigens for the treatment of autoimmune diseases or disorders as described herein. The invention also involves the use of anti-CD3 antibodies and self-antigens in the manufacture of medicaments for treating autoimmune diseases or disorders as described herein.

The invention also provides kits relating to the methods of the invention. For example, in one aspect, a kit can comprise: (a) an anti-CD3 antibody; (b) a self-antigen; and (c) instructions for coadministration of the anti-CD3 antibody and the self-antigen comprising a dosing schedule and dosing amounts for the anti-CD3 antibody and the self-antigen. In another aspsect, a kit can comprise: (a) an anti-CD3 antibody; (b) an islet cell associated antigen; and (c) instructions for coadministration of the anti-CD3 antibody and the islet cell associated antigen comprising a dosing schedule and dosing amounts for the anti-CD3 antibody and the islet cell associated antigen.

### BRIEF DESCRIPTION OF THE DRAWINGS

FIG. 1. Reestablishment of euglycemia after various doses of anti-CD3 Fab′2 in the NOD mouse model for T1D. NOD mice were treated after recent onset of diabetes with 5 i.v. injections of anti-CD3 F(ab′)2 alone. (See Example 1.) FIG. 1A shows that five 10 μg does of anti-CD3 provides a transient protection. FIG. 1B shows that five 50 μg does of anti-CD3 provides a partial protection (20%). FIG. 1C shows that five 100 μg doses of anti-CD3 provides 50% protection. FIG. 1C also shows a therapeutic window (shaded area and bidirectional arrow) determined by BGV (250-500 mg/dl) at the time of first anti-CD3 administration during which reversion of T1D (Type I diabetes) occurs. BGV mg/dl represents the blood glucose value. BGV values exceeding 250 mg/dl are considered diabetic or unprotected. For the combinatorial studies with antigen (see FIGS. 2 and 3, and Example 1), the 100 μg dose of anti-CD3 was employed since this appeared to offer a therapeutic window for further improvement by coadministration.

FIG. 2A. Clear synergistic effect of anti-CD3 and self-antigen immunization. Anti-CD3 and islet self-antigens (either proinsulin or GAD self-antigens) were coadministered to NOD mice with recent-onset T1D. NOD mice were treated with five 100 Mg anti-CD3 F(ab′)₂ i.v. doses in combination with four 100 μg doses of islet-specific antigens rhGAD65 or with four 40 μg doses of mouse proinsulin II peptide without the CTL epitope. The coadministration of anti-CD3 and proinsulin provided a 100% protection (n=4), and the coadministration of anti-CD3 and GAD provided a 75% protection (n=4). Mice with blood glucose values exceeding 250 mg/dl were considered diabetic and therefore unprotected. The incidence of diabetes, after each treatment, is summarized. The number of mice in each group is indicated (n=4). None of the mice received insulin at any time during the experiment. Anti-CD3 treatment was given 5 consecutive days at 100 μg/day when blood glucose reached/exceeded 250 mg/dl. Each protocol is described in Example 1 (see groups 1, 11 and 13). Thus, treatment with anti-CD3 and intransal proinsulin resulted in 100% protection compared to 50% protection with anti-CD3 alone (see FIG. 1C), and no protection with proinsulin antigen administered alone given to recent-onsest diabetic NODs (not shown).

FIG. 2B. Enhanced remission of diabetes when anti-CD3 mAb (monoclonal antibody) is combined with antigen. Female NOD mice, found to have diabetes (diagnosed when the glucose level was >200 mg/dl) were treated with F(ab′)2 fragments of anti-CD3 mAb (145-2C11, n=9), proinsulin peptide (Auspep, n=8), or both F(ab′)2 fragments of anti-CD3 mAb and proinsulin peptide (n=8). The dose of proinsulin peptide used was 40 μg i.n. on days 0, 1, 7, 12 and the dose of the F(ab′)2 fragments of anti-CD3 mAb used was 50 μg i.v. on days 0-4. The blood sugar levels were measured using a hand held glucose meter 2-3×/week daily for 7 weeks. Mice that were found to have a glucose level>200 mg/dl were scored as having diabetes. Treatment with the combination of the F(ab′)2 fragments of anti-CD3 mAb with proinsulin peptide

FIG. 3. Incidence of diabetes after anti-CD3 and/or antigen-specific treatments in RIP-LCMV-GP mice. FIG. 3A shows experiments with RIP-LCMV-GP mice that were treated with anti-CD3 F(ab′)2 and human GAD65 (by using an eukaryotic expression plasmid [pCMV/hGAD65]), alone or in combination. Mice with blood glucose values exceeding 250 mg/dl were considered diabetic. Anti-CD3 treatment was given 5 consecutive days (days 15 to 20 after LCMV infection). Each protocol is described below in Example 1 Section D (see groups 1, 6, 12 and 14). FIG. 3B shows experiments with RIP-LCMV mice (GP and NP) that were treated with anti-CD3 F(ab′)2 for 5 days (100 μg/day i.v.). The incidence of diabetes was compared between two groups distinguished according to blood glucose values measured before the first anti-CD3 injection (BG<or >500 mg/dl, left or right panel respectively of FIG. 3B).

FIG. 4 shows staining of a RIP-LCMV mouse islet day 10 after LCMV infection for MHC class I restricted LCMV lymphocytes specific for the LCMV (self) transgene expressed in beta cells. Few driver clones are necessary for inducing disease rapidly in this model, as it is usually observed in RIP-LCMV-GP mice (2 weeks after LCMV infection). These CTL are essential for diabetes, because disease does not occur after infection with LCMV-GP₃₃ or NP₃₉₆ viral escape variants. Negative control stains of MHC mismatched sections did not show any tetramer positive cells. Control sections of LCMV-GP TcR transgenic spleens showed 80-90% positive cells as expected (positive control).

FIG. 5 depicts a basic schematic of the RIP-LCMV model for autoimmune diabetes. RIP-LCMV transgenic mice express a well-defined target autoantigen exclusively in pancreatic β-cells but not any other organs. Specifically, the RIP-LCMV transgenic mice express the nucleoprotein of Lymphocytic Choriomeningitis virus (LCMV) under the control of the insulin promoter (RIP) in the pancreatic beta cells.

FIG. 6 depicts a model of the cellular interactions in the setting of FcR non-binding anti-CD3 mAb. Both regulatory and antigen (gold) reactive effector cells (green) may be affected by FcR non-binding anti-CD3 mAb. T regulatory cells (CD4+CD25+cells (red)) may be stimulated by the mAb to secrete IL-10 and/or TGF-b. The specificity of the T regulatory cells is not known. In addition, human studies suggest an effect of anti-CD3 mAb on CD8+ cells (blue) but the interactions between CD8+ and CD4+ cells are not well described.

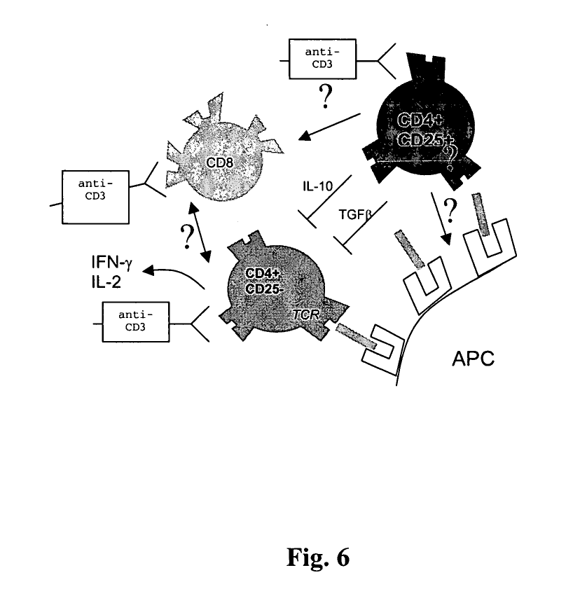

FIG. 7 depicts a model of the effects of antigen on T regulatory cells. Immunization with islet antigens induces T regulatory cells that are CD4+ and produce IL-4 and IL-10. These calls can prevent T1DM in recipients, when transferred during the pre-diabetic stage and after recent-onset T1D in some investigations. They block augmentation of autoaggressive responses as bystander suppressors acting in the pancreatic draining lymph node.

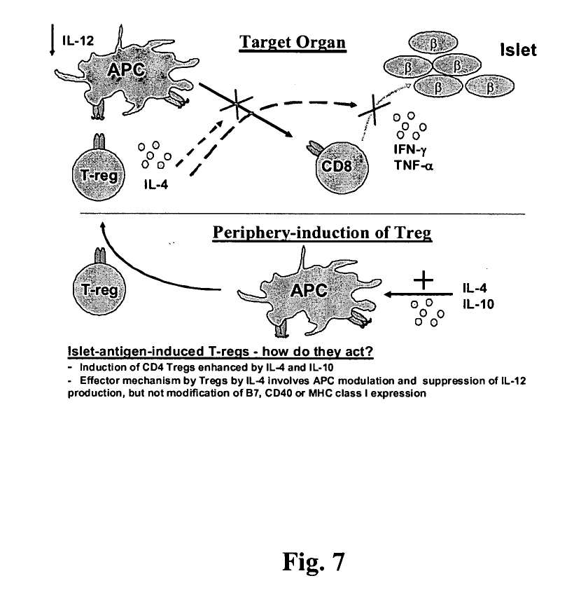

FIG. 8 shows the effects of treatment with hOKT3γ1(Ala-Ala) on C-peptide responses to a MMTT (mixed-meal tolerance test) over 2 years. The mean+SEM (standard error of the mean) of the group responses to MMTT's done at 6 month intervals are shown. (p<0.001 by RMANOVA (repeated measures analysis of variance).) The average responses are significantly different after 24 months (p<0.01).

FIG. 9A (before mAb treatment) and FIG. 9B (after mAb treatment) show the induction of CD4+ IL−10+cells in vivo by treatment with hOKT3γ1(Ala-Ala). Cells were isolated from patients after treatment with the mAb and stained for intracytoplasmic IL-10 and IFN-γ without further activation ex vivo. IL-10+CD4+ cells were identified in 5/6 patients within 1 week after the last dose of mAb.

FIGS. 10A and 10B shows induction of regulatory T cells in vitro with anti-CD3 mAb. See text in Example 4 for details of the experimental procedure. FIG. 10A shows the proliferation results of cells cultured in PHA (phytohaemagglutinin) as compared to anti-CD3 mAb followed by IL-10/IL-2. In the left panel of FIG. 10A, responder cells alone were stimulated with PHA and the percentage of proliferating cells was 67%. In the right panel of FIG. 10A, responder cells were stimulated with PHA in the presence of cells treated with anti-CD3 mAb/IL-10/IL-2, and the percentage of proliferating cells was 43%. The number of proliferating responder cells (i.e. diluted CFSE (carboxy-fluorescein diacetate, succinimidyl ester)) in the presence of the added cells is shown. FIG. 10B shows that cells cultured with IL-110/IL-2 alone (with anti-CD3) did not show the same inhibitory effect. In the left panel of FIG. 10B, responder cells alone were stimulated with PHA and the percentage of proliferating cells was 50%. In the right panel of FIG. 10B, responder cells were stimulated with PHA in the presence of IL-10/IL-2 cultured cells, and the percentage of proliferating cells was 54%.

FIG. 11A and FIG. 11B shows the inhibitory properties of cells grown in hOKT3γ1(Ala-Ala) and IL-10/IL-2. PBMC (peripheral blood mononuclear cells) were stimulated with hOKT3γ1(Ala-Ala) as described in Example 4 (FIG. 11A, sorted into CCR4+ or—subsets and then cultured with IL-10/IL-2). Other cells were cultured in IL-10/IL-2 (FIG. 11B), sorted, and then cultured for an addition 19 days in IL-10/IL-2. Both groups of cells were added to fresh PBMC. Uptake of 3H-thymidine was measured 72 hrs after the addition of PHA.

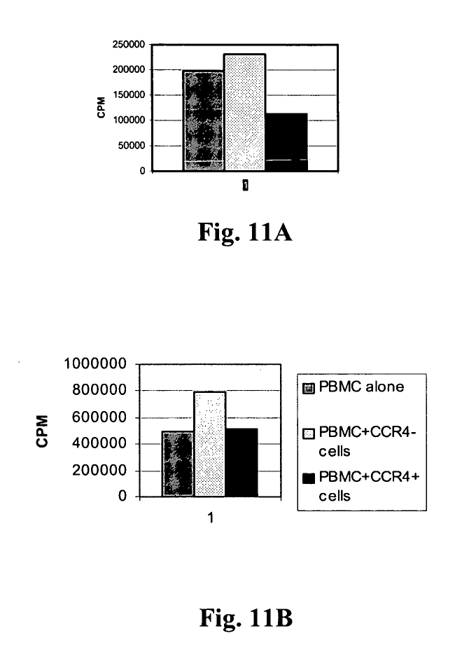

### DETAILED DESCRIPTION OF THE INVENTION

Previous attempts to induce immune regulation in autoimmune diseases by administering defined antigens or anti-CD3 antibodies have shown efficacy, but the duration of these effects or their efficacy after disease presentation are limitations for bringing these approaches into the clinic. The present invention can provide a novel strategy for treatment of autoimmune diseases in which antigen and anti-CD3 mAb are coadministered. The administration of these agents together provides a synergistic protective effect, where the co-administration can at least alter the response to self-antigens, induce a non-pathogenic response to self-antigens, and induce local immune regulation.

### Terms

As used herein, “coadministration” refers to administering anti-CD3 and antigens so that their dosing regimens overlap. The anti-CD3 antibodies and the antigens do not need to be administered at the same time. For example, in a non-limiting embodiment, they can be administered in a regimen where they are encountered at least at one time point essentially simultaneously by the individual's immune system. Thus, for example, on day 1 of treatment, both anti-CD3 and antigen can be administered, and following day 1, anti-CD3 and antigen can be administered on different days.

The term “antibody” as used herein, unless indicated otherwise, is used broadly to refer to both antibody molecules and a variety of antibody derived molecules. Such antibody derived molecules comprise at least one variable region (either a heavy chain of light chain variable region) and include, but are not limited to, molecules such as Fab fragments, Fab′ fragments, F(ab′)2 fragments, Fd fragments, Fabc fragments, Fd fragments, Fabc fragments, Sc antibodies (single chain antibodies), diabodies, individual antibody light chains, individual antibody heavy chains, chimeric fusions between antibody chains and other molecules, and the like.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site. Thus, an intact IgG antibody has two binding sites. Except in bifunctional or bispecific antibodies, the two binding sites are the same.

The chains all exhibit the same general structure of relatively conserved framework regions (FR) joined by three hyper variable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia et al., J. Mol. Biol. (1987) 196:901-917; Chothia et al. Nature (1989) 342:878-883.

The term “variable region” as used herein in reference to immunoglobulin molecules has the ordinary meaning given to the term by the person of ordinary skill in the art of immunology. Both antibody heavy chains and antibody light chains may be divided into a “variable region” and a “constant region”. The point of division between a variable region and a heavy region may readily be determined by the person of ordinary skill in the art by reference to standard texts describing antibody structure, e.g., Kabat et al., “Sequences of Proteins of Immunological Interest: 5th Edition” U.S. Department of Health and Human Services, U.S. Government Printing Office (1991).

As used herein, the term “humanized” antibody refers to a molecule that has its CDRs (complementarily determining regions) derived from a non-human species immunoglobulin and the remainder of the antibody molecule derived mainly from a human immunoglobulin.

A “bispecific” or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai et al., Clin. Exp. Immunol. (1990) 79: 315-321; Kostelny et al, J. Immunol. (1992) 148:1547-1553. In addition, bispecific antibodies may be formed as “diabodies” (Holliger et al. PNAS USA (1993) 90:6444-6448) or “Janusins” (Traunecker et al. EMBO J. (1991) 10:3655-3659 and Traunecker et al. Int. J. Cancer Suppl. (1992) 7:51-52). Bispecific antibodies do not exist in the form of fragments having a single binding site (e.g., Fab, Fab′, and Fv). In the present methods, the anti-CD3 antibody should be at least “bivalent,” or in other words, it should have at least two antigen binding sites that have the same binding specificity.

As used herein, the term “islet cell antigen” refers to antigens that can come from the pancreatic islets of Langerhans, which can be divided into four main cell types: alpha, beta, delta and gamma cells. Specific examples of islet cell antigens include, but are not limited to, islet cell antigen (ICA) 512/IA2, islet cell antigen p69 (ICA69), glutamic acid decarboxylase (GAD65), islet-specific glucose-6-phosphatase catalytic subunit-related protein (IGRP), insulin, proinsulin, and derivatives thereof. Alternative names for ICA512/IA2 are, PTPRN2, IA-2, ICA512, R-PTP-N, IA-2/PTP, PTPIA2, islet cell antigen 2, islet cell antigen 512, islet cell autoantigen 3, protein tyrosine phosphatase-like N, protein tyrosine phosphatase receptor pi, phogrin, tyrosine phosphatase IA-2 beta, IAR/receptor-like protein-tyrosine phosphatase.

In the present invention, the term “non-mitogenic” is used to describe certain non-limiting types of anti-CD3 antibodies that do not cause T-cell proliferation.

In the invention, the term “autoantigen” refers to a self-antigen that is a target of immune responses. Such immune responses against the self-antigen or autoantigen can be described as autoaggressive responses (or called autoimmune responses), and include responses that are pathogenic.

In the invention, a T cell receptor is specific for an antigen or has a specificity to the antigen in reference to the specific binding of a T cell receptor to the antigen as presented by a MHC (major histocompatibility) molecule.

The invention assumes the understanding of conventional molecular biology methods that include techniques for manipulating polynucleotides that are well known to the person of ordinary skill in the art of molecular biology. Examples of such well known techniques can be found in Molecular Cloning: A Laboratory Manual 2nd Edition, Sambrook et al., Cold Spring Harbor, N.Y. (1989). Examples of conventional molecular biology techniques include, but are not limited to, in vitro ligation, restriction endonuclease digestion, PCR, cellular transformation, hybridization, electrophoresis, DNA sequencing, and the like.

The invention also assumes the understanding of conventional immunobiological methods that are well known to the person of ordinary skill in the art of immunology. Basic information and methods can be found in Current Protocols in Immunology, editors Bierer et al., 4 volumes, John Wiley & Sons, Inc., which includes teachings regarding: Care and Handling of Laboratory Animals, Induction of Immune Responses, In Vitro Assays for Lymphocyte Function, In Vivo Assays for Lymphocyte Function, Immunofluorescence and Cell Sorting, Cytokines and Their Cellular Receptors, Immunologic Studies in Humans, Isolation and Analysis of Proteins, Peptides, Molecular Biology, Biochemistry of Cell Activation, Complement, Innate Immunity, Animal Models for Autoimmune and Inflammatory Disease (which includes chapters on the NOD mouse model, the SLE mouse model (for lupus), and induction of autoimmune disease by depletion of regulatory T cells), Antigen Processing and Presentation, Engineering Immune Molecules and Receptors, Ligand-Receptor Interactions in the Immune System, Microscopy, and Abbreviations and Terminology for common immune system genes and proteins, including the CD system for Leukocyte Surface Molecules.

### Overview

Studies concerning the effects of anti-CD3 antibodies suggest that the antibodies can alter autoimmune responses towards a non-pathogenic phenotype possibly involving the action of regulatory T cells. Initial studies with anti-CD3 mAb (hOKT3γ1(Ala-Ala)) in patients with new onset T1D disease have shown that the drug can induce functional immunologic tolerance that lasts for at least 1 year after treatment. However, follow up studies of patients after the first year of disease has shown that there is a statistically significant improvement in insulin secretion even 2 years after treatment with anti-CD3 mAb, but there is a decrease in the C-peptide response after the first year to 71% of the baseline response. These findings suggest that tolerance may diminish after the first year following treatment and that an additional intervention is needed to sustain the clinical response.

Therefore, an underlying rationale behind the present methods is that the administration of self-antigens identified as targets of an autoimmune response (especially self-antigens that are targets of T-cell dependent autoimmune responses) together with anti-CD3 antibodies can alter the response to those self-antigens and prevent progression of autoimmunity. By rechallenging with the autoantigens and stimulating the non-pathogenic response, the blockade of the autoimmune process can be maintained. Without being bound by theory, it is believed that the coadministration of anti-CD3 and self-antigens can reestablish tolerance to those self-antigens, and also other self-antigens that are targets to the particular autoimmune disorder in question. Preclinical evidence provided herein shows that the combination of anti-CD3 and autoantigen is synergistic in reversing autoimmune diabetes, and therefore, coadministration has the potential to provide synergistic protection in reversing other autoimmune disorders.

Beyond its potential synergistic effects, another advantage of coadministration of anti-CD3 and self-antigens relates to the problem that autoimmune disorders often include autoaggressive responses against multiple self-antigens. It may be difficult to attempt to anergize or delete all of the autoaggressive lymphocytes using direct antigen-specific tolerization with all of their cognate antigens, because all of the cognate antigens may not be known. For example, Type 1 diabetes (T1D) is thought to be caused by autoaggressive lymphocytes that enter the islets of Langerhans, where they destroy β-cells. Activation of such cells is probably multi-factorial involving a genetic predisposition, environmental triggers such as viruses and maybe damage to the pancreas (islets cells), for example caused by a local pro-inflammatory reaction. Since the autoaggressive process is usually fairly advanced when pre-diabetic human individuals are identified by screening for islet-cell antibodies, one can assume that aggressive responses to more than one islet-antigen will be ongoing during this stage of the disease. Furthermore, chronic non-specific systemic immune suppression is not considered an option, since diabetes frequently affects young individuals and life long immune suppression is associated with side effects that are unacceptable compared to even insulin therapy alone. Therefore, a curative immune-based intervention with specificity and low systemic side effects is very desirable. The present methods circumvent the problem of multiple self-antigenic targets, because the coadministration of anti-CD3 and a single self-antigen may be sufficient to re-establish tolerance to multiple self-antigens that are targets in an autoimmune disorder. The present methods also circumvent the problems of chronic non-specific systemic immune suppression, because the coadministration of anti-CD3 and self-antigens can reestablish long-term tolerance without the need for continuous life-long dosing.

Without being bound by theory, the invention provides that the coadministration of anti-CD3 and self-antigens has the potential to synergistically establish long-term tolerance in part by inducing the activation/expansion of regulatory T-cells (and also regulatory antigen presenting cells (APCs)). In murine systems, regulatory T cells have the capacity to control autoimmune disease. Some cells appear to act in a systemic non antigen-specific way, such as the CD25+ positive lymphocytes that are the focus of many laboratories' efforts. Belghith et al., Nat Med (2003) 9:1202-8; Chatenoud et al., Immunol Rev (2001) 182:149-63; Green et al., Proc Natl Acad Sci USA (2003) 100:10878-83; Asseman et al., Autoimmun Rev (2002)1:190-7. These cells are found in decreased numbers in several autoimmune-prone conditions in mice. For example, the accelerated diabetes that occurs in CD28^{-/-} NOD mice is due to the absence of regulatory CD4+ CD25+T cells and can be reversed by transfusion of these cells.

A number of different phenotypes of regulatory T cells have been described. They can arise after thymectomy and can be induced after systemic immune modulation with co-stimulation blockers or FcR non-binding anti-CD3 (further described herein). Their effector functions are not fully known. They appear to be part of the immune system's intrinsic balance and their loss results in severe immune dysregulation and autoimmunity. Th2-like regulators with defined antigen specificity have been described. They are thought to act as bystander suppressors and arise after antigen-specific immunization. Homann et al., J. Immunol. (1999) 163:1833-8. Depending on their effector function they have been termed Th3 (TGF-β producers). These cells are antigen specific lymphocytes with specialized effector functions and do not behave like Th2 cells. Applying the so-called Th1/Th2 paradigm to these cells can therefore be misleading.

Subpopulations of CD8+ regulatory T cells have also been described in human and mouse systems. One report has suggested that a subpopulation of CD8+ CD28 low cells can mediate transplant tolerance by interaction with the molecule ILT3 on antigen presenting cells. Another cell type appears to regulate CD4+ T cells by recognition of non-classical Class I MHC molecules (Qa-1 or HLA-E) that are expressed on activated CD4+ cells. Colovai et al., Transplant Proc (2001) 33:104-7; Liu et al., Transplant Proc (2001) 33:82-3; Chang., et al., Nat Immunol (2002); Jiang, H., et al., Annu Rev Immunol (2000) 18:185-216. Lastly, APCs with active regulatory function have recently been described. Homann et al., Immunity (2002) 16:403-15; Serreze et al., Curr Dir Autoimmun (2003) 6:212-27; Boudaly et al., Eur Cytokine Netw (2002) 13:29-37. These can arise after blockade of costimulation, contact with anergic T cells or regulatory cells or other immune modulations. They are of interest, because of their antigen specific regulatory effector functions. Thus, the analysis and tracking of these ‘regulatory cells’ in respect to co-administration by anti-CD3 and self-antigen may provide important insights into the mechanisms underlying the induction or reestablishment of tolerance.

As mentioned above, the present methods circumvent the problem of multiple self-antigenic targets, because the co-administration of anti-CD3 and a single self-antigen may be sufficient to re-establish tolerance to multiple self-antigens that are targets in an autoimmune disorder. Without being bound by theory, the present methods might reestablish tolerance to multiple self-antigens by a process called “bystander suppression,” over and above the mechanisms of deletion of autoaggressive cells and of antigen specific anergy.

Bystander suppression relates to the phenomenon of antigenic spreading. Antigenic spreading is thought to be an essential component during the progression of local autoimmune processes. One can therefore assume that when patients have several autoantibodies, the autoaggressive response may involve many self-antigens (or “autoantigens”). Since a majority of the autoantigens might not be identified for a particular autoimmune disorder, it is not possible to tolerize each autoaggressive specificity with a therapeutic regimen that involves knowledge of the respective MHC restriction element and peptide. The induction of regulatory cells by the present methods has several advantages in this situation. It is known for example, that regulatory T cells in T1D can act locally in the PDLN and islets as bystander suppressors, which means that they can suppress aggressive lymphocytes with other auto-antigenic specificities. This can occur by modulating antigen presenting cells (APCs), for example, by secretion of cytokines with immune modulatory function. Thus, such bystander suppressor T regulatory cells can dampen autoaggression to several other autoantigens without knowing their precise specificity. Since anti-CD3 creates a systemic immune deviation involving also the upregulation of IL-10, it is possible that antigen specific immunization (i.e., administration of self-antigens) during anti-CD3 administration will have a higher likelihood of inducing T-regulatory cells that can then act as bystander suppressors.

Not all components of an ongoing auto-reactive process are necessarily damaging to the targeted organ. Indeed, auto-aggressive and auto-reactive regulatory responses have been described in many experimental models for autoimmune diseases. These co-exist in a relatively fragile equilibrium, which usually shifts in favor of the aggressive response before clinically manifested autoimmunity develops. Shifting the phenotype of the pathogenic immune response, and/or enhancing the regulatory component by a well-chosen immunomodulation should be of great interest to block and reverse the course of the disease. Without being bound by theory, the present invention provides that the coadministration of antigen and anti-CD3 antibodies will alter the response to the antigen so that the response will be non-pathogenic and/or that regulatory T cells induced by the combination therapy will modify the responses to the antigen and prevent autoimmunity, and that the effect of the coadministration is synergistic in reestablishing/inducing tolerance or in reducing deleterious T-cell mediated autoimmune effects. Thus, a non-limiting rationale of this invention is that the anti-CD3 antibody is able to ‘reset’ the immune system, enabling some antigen specific immunizations to induce regulation that can maintain long-term tolerance.

### Anti-CD3 Antibodies

Prior to the invention, there has been no evidence that the combined administration of anti-CD3 and self-antigen could result in a synergistic effect of inducing or reestablishing tolerance for autoantigens. Further, such a synergistic result is by no means expected, as it is quite possible that the immunosuppressive capabilities of anti-CD3 can nullify any proactive effects that the administration of self-antigens might have toward establishing tolerance to these antigens.

Thus, the present methods involve the coadministration of anti-CD3 antibodies and self-antigens that are targets of autoimmune T-dependent responses. Specific examples of self-antigens contemplated by the invention are described below. This section provides specific examples of anti-CD3 antibodies that may be used in the present methods.

Generally, the present methods contemplate the use of any anti-CD3 antibody that in conjunction with self-antigen administration is capable of inducing or reestablishing tolerance to the self-antigen. One qualification to this general use of anti-CD3 antibodies is that the antibodies should not be in monomeric form, or in other words, antibodies of the present invention should possess at least two-antigen binding sites. Therefore, Fab anti-CD3 antibodies generally do not work in the present invention unless single Fab molecules are joined together. The present methods can include the use of anti-CD3 antibodies that are full-length or that are multimeric fragments thereof. Multimeric antibody fragments can include, for example, F(ab′)₂, bivalent antibodies including single chain bivalent antibodies, biabody antibodies, and bivalent single chain Fv antibodies. The antibodies can be any class of antibody, i.e., IgG, IgM, IgE, IgA and IgD. The antibodies can be of any subclass, for example, for human antibodies: IgG1, IgG2, IgG3, IgG4, IgA1, and IgA2, and for mouse antibodies: IgG1, IgG2a, IgG2b.

The anti-CD3 antibodies can be polyclonal or monoclonal. The antibodies can also be chimeric (i.e., a combination of sequences from more than one species, for example, a chimeric mouse-human immunoglobulin), humanized or fully-human. Human antibodies avoid certain of the problems associated with antibodies that possess murine or rat (or other species) variable and/or constant regions. The presence of such murine or rat derived proteins can lead to the rapid clearance of the antibodies or can lead to the generation of an immune response against the antibody by a patient. In order to avoid the utilization of murine or rat derived antibodies, one can develop humanized antibodies or generate fully human antibodies through the introduction of human antibody function into a rodent so that the rodent would produce antibodies having fully human sequences. For example, U.S. Pat. Nos. 5,770,429; 6,150,584; and 6,677,138 relate to transgenic mouse technology, i.e., the HuMAb-Mouse™ or the Xenmouse®, to produce high affinity, fully human antibodies to a target antigen.

In one embodiment, the anti-CD3 antibody does not bind to Fc Receptors (FcR). Such anti-CD3 antibodies are denoted herein as “FcR non-binding anti-CD3 Ab.” One particular FcR non-binding anti-CD3 Ab that can be used is the OKT3 antibody. The invention also includes the use of mutants or variants of the OKT3 antibody, including hOKT3γ1(Ala-Ala) and hOKT3 γ3 (IgG3) (Herold, K. et al., N. Engl. J. Med. (2002), 346: 1692-8; Xu, D. et al., Cell Immunol. (2000), 200: 16-26). hOKT3γ1(Ala-Ala) exhibits similar functions in the mouse compared to humans and has a mutated Fc-binding region. It is non-mitogenic but induces signaling in T cells. Anti-CD3-IgG3 is similar to the Ala-Ala version, as this antibody exhibits similar functions in the mouse compared to humans and has a mutated Fc-binding region. It is also non-mitogenic, but also induces signaling in T cells.

Further, anti-CD3 Fab′2 antibodies are contemplated, such as the antibody that is derived from the mouse 2C11 cell clone—it is FcR non-binding, non-mitogenic and induces signaling in T cells. Methods relating to the use and production of anti-CD3 antibodies, including OKT3 antibodies and variants/mutants thereof, are described in U.S. Pat. Nos. 6,113,901; 6,491,916; and 5,885,573, which are hereby incorporated by reference. Further, in one embodiment of the invention, the anti-CD3 antibodies are not immune depleting.

Anti-CD3 antibodies can be administered in an amount from about 5 μg to about 2000 μg. The administration can be daily for a period of about 1-14 days, for example. In one embodiment, the administration is daily for a period of 10 days. In another embodiment, the administration is daily for a period of 12 days. In another embodiment, the anti-CD3 antibody is administered on day 1 in an amount of about 200-250 μg/m², on day 2 in an amount of about 400-500 μg/m², and on days 3-12 in an amount of about 900-1000 227 μg/m². The administration should be intravenous (i.v.). For T1D, anti-CD3 antibodies can be administered, for example, on days 0-10 post onset of hyperglycemia.

### Self-Antigens

The present invention includes methods for treating autoimmunity and/or establishing or inducing tolerance by the coadministration of anti-CD3 antibodies and self-antigens that are the target of T-dependent autoimmune responses. Besides autoimmune diseases, present methods may also be used to establish tolerance to allergens, where allergenic peptides or proteins are coadministered with anti-CD3 antibodies.

Non-limiting examples of autoimmune diseases that are mediated by T-cells contemplated for treatment by the present methods include, but are not limited to, the diseases provided in the Table below (where self-antigens relevant to the disease are also included, such that these self-antigens can be coadministered with anti-CD3 antibodies):

TABLE 1
Autoimmune Diseases and Self-Antigen Targets

| Autoimmune Disease | Self-Antigen |
|---|---|
| Graves' Disease | Thyroid-stimulating hormone |
| | receptor |
| Hashimoto's thyroiditis | Thyroglobulin, thyroid peroxidase, |
| | thyroid antigens |
| Hypoglycemia | Insulin receptor |
| Multiple sclerosis | MBP (myelin basic protein; Steinman |
| | et al., Mol. Med. Today (1995) 1: 79; |
| | Warren et al., Proc. Natl. Acad. |
| | Sci. USA (1995) 92: 11061 |
| | PLP, trnasaldolase, 2′, 3′ cyclic |
| | nucleotide 3′ phosphodiesterases |
| | (CNP), MOG and MAG (Steinman, |
| | L. Nature (1995) 375: 739 |
| Mixed essential cryoglobulinemia | |
| Systemic lupus erythematosus | DNA, histones, ribosomes, snRNP, |
| | scRNP |
| Type I Diabetes | GAD65 (glutamic acid decarboxlyase |
| | 65; Baekkeskov et al., Nature (1990) |
| | 347: 151) |
| | Insulin (Palmer et al., Science (1983) |
| | 222: 1337); including the B9-23 |
| | peptide comprising amino acids |
| | 9-23 of the insulin B chain (Daniel |
| | et al., Proc. Natl. Acad. Sci. USA |
| | (1995) 93: 956-960; Wong et al., |
| | Nat. Med. (1999) 5: 1026-1031) |
| | For an alignment of insulin sequences |
| | between species, see Table I in |
| | Homann et al., J. Immunol. (1999) |
| | 63: 1833-1838. |
| | Proinsulin; including the B24-C36 |
| | peptide comprising amino acids |
| | 24-36 spanning the proinsulin |
| | B-chain C-peptide junction (Chen et |
| | al., J. Immunol. (2001) 167: |
| | 4926-4935; Rudy et al., Mol. Med., |
| | (1995) 1: 625-633) |
| | HSP60 (heat shock protein 60); |
| | ICA512/IA-2 (islet cell antigen 512; |
| | Rabin et al., J. Immunol. (1994) 152: |
| | 3183) |
| | see also Table 2 in Example 1 |

Thus, in certain embodiments, the present invention provides methods for treating autoimmunity or for inducing/reestablishing tolerance that involves the coadministration of an anti-CD3 antibody and self-antigens such as those listed in the Table above. The administered self-antigens can be in the form of the whole protein or peptide fragments thereof. The sequence of the protein or peptide fragments can be wild-type or mutant. Further, the protein or peptide can be introduced into a subject as a protein or peptide in a pharmaceutically acceptable carrier or the protein or peptide can be encoded by an expression vector, where the expression vector is introduced (for example, see the Examples where the self-antigen is expressed in a subject by a pCMV-expression vector). For further description regarding the administration of self-antigens via their expression vectors, (and for specific antigens that can be administered) see U.S. Patent Publication US 2002/0107210, which is hereby incorporated by reference.

Generally, antigens can be administered in an amount from about 100 μg to about 2000 μg per kg body weight, for example. Antigens can be administered on a dosing schedule comprising multiple days, where the total amount of antigen administered, in one embodiment, is about 100 μg to about 2000 μg. In one embodiment, the antigen is administered in an amount of about 50 to about 200 μg, daily for four days. With regards to coadministration with anti-CD3, anti-CD3 and antigen can both be administered, for example, on day 1, and following day 1, the dosing times may differ. Further, after the initial dosing regimen of anti-CD3 and antigen, both anti-CD3 and antigen, anti-CD3 alone, or antigen alone, can be administered as a booster. For example, after initial dosing, the booster can be administered at a time from about 6 months to about 2 years after initial dosing. The antigens can be administered intranasally, subcutaneously (s.c.), intramuscularly (i.m.), or intravenously (i.v).

As listed in the Table above, examples of antigens that can be coadministered with anti-CD3 for the treatment of diabetes (reestablishing tolerance in T1D patients) include, for example, insulin, proinsulin, GAD65, ICA512/IA-2 and HSP60. The antigens can be initially given either after the onset of hyperglycemia, for example, within 2 months, within 1-2 months, within 6 weeks, or following a 10-day delay. Unlike the “therapeutic window” observed for mice in Example 1, administration of self-antigens for T1D does not have to occur within specific blood glucose value levels of the human patient.

In one embodiment for the treatment of T1D, 100 μg of antigen can be administered on each of four different days. The antigen can be administered in alum and injected s.c. for example. In another embodiment, the antigen can be administered intranasally in an amount from about 1 to about 2 mg on each of four different days. In another embodiment, the antigen can be administered intranasally in an amount of about 1.5 mg on each of four different days. The effects of treatment on reversal of diabetes can be studied by measurement of glucose levels and by evaluation of insulitis.

In one embodiment, human insulin or human insulin analog X38 (NovoNordisk) can be administered. It can be administered, for example, at an amount of about 0.05 mg/dose in 10 μl PBS on each of four separate days for initial dosing. In another embodiment, porcine insulin-B chain can be administered, for example, at an amount of about 5 mg/kg s.c. in 100 μl. A modified peptide can also be administered that has slower absorption and does not induce the anaphylaxis that has been seen with insulin B9-23 peptide alone.

In one embodiment, porcine insulin-B chain APL (altered peptide ligand) can be administered. It can be administered, for example, at an amount of about 5 mg/kg, s.c. in 100 μl, at days 0, 3, 7, 10 and 15 of treatment. The antigen can be obtained commercially from Neurocrine, San Diego, Calif., USA, (Alleva et al., Diabetes (2002) 51:2126-34).

In one embodiment, human GAD 65 protein (hGAD65) can be administered. It can be administered, for example, at an amount of about 100 μg s.c. in 300 μl PBS at days 0, 1, 7, and 12. It can be obtained commercially from Diamyd, Stockholm, Sweden.

In one embodiment, the proinsulin II peptide can be administered, either with or without CTL epitope (proinsulin peptide).

In one embodiment, HSP60 peptide (DiaPep277) can be administered (Raz et al., Lancet (2001), 358:1749-53).

### T-Regulatory Cells

T regulatory cells may be the basis behind long lasting tolerance initiated by the administration of anti-CD3 and antigen. It is possible that after the initial coadministration, further antigen administration may help to maintain the antigen-specific T regulatory cell population for long-time periods. Thus, the invention also provides methods for treating autoimmunity or inducing/establishing tolerance by administering anti-CD3 antibodies and self-antigen in order to expand or produce the population of T regulatory cells that have an antigen specificity toward the self-antigen. The coadministration can be used to expand or produce T regulatory cells in vivo. The invention also includes the isolation of T regulatory cells after coadministration, where the isolated cells can be further expanded/grown in vitro. (See Examples for methods of isolation and expansion.) In addition to expansion in vitro, the T regulatory cells can be further exposed to anti-CD3 and antigen. After expansion in vitro, the regulatory cells can be frozen-down for future use in the patient or readministered into the patient.

In one embodiment, the isolated T regulatory cells are at least CD4+ and CD25+. In another embodiment, the isolated T regulatory cells are at least CD4+, CD25+, GITR+, CTLA-4+ and CD62L+. In another embodiment, the isolated T regulatory cells are at least CD4+, IL-10+, and TGF-β+. In another embodiment, the isolated T regulatory cells are at least CD4+, IL-10+, CD45RO+, and CCR4+. In another embodiment, the isolated T regulatory cells are at least CD4+, IL-10+, CD45RO+, CCR4+, and TGF-β+. In another embodiment, the isolated T regulatory cells are at least CCR4+, CD62L+, and CD45RO+. In another embodiment the isolated T regulatory cells are at least CD4+ and IL-4+. In another embodiment, the isolated T regulatory cells are at least CD8+.

As various changes can be made in the above methods and compositions without departing from the scope and spirit of the invention as described, it is intended that all subject matter contained in the above description, shown in the accompanying drawings, or defined in the appended claims be interpreted as illustrative, and not in a limiting sense.

### EXAMPLES

The following examples are representative of techniques employed by the inventors in carrying out aspects of the present invention. It should be appreciated that while these techniques are exemplary for the practice of the invention, those of skill in the art, in light of the present disclosure, will recognize that numerous modifications can be made without departing from the spirit and intended scope of the invention. Thus, the examples described below are provided to illustrate the present invention and are not included for the purpose of limiting the invention.

### Example 1

### Experimental Results of Anti-CD3 and Antigen-Specific Immunotherapy to Treat Recent Onset Diabetes in Mice

The experimental results described below were obtained using the experimental methods in the section “Methodology and Experimental Plans.”

NOD model: Published studies of anti-CD3 mAb treatment of diabetic mice have shown remarkable efficacy (generally 80%) in permanent reversal of diabetes (Chatenoud et al., Proc Natl Acad Sci USA (1994) 91:123-7; Chatenoud. et al., J Immunol (1997) 158:2947-54).

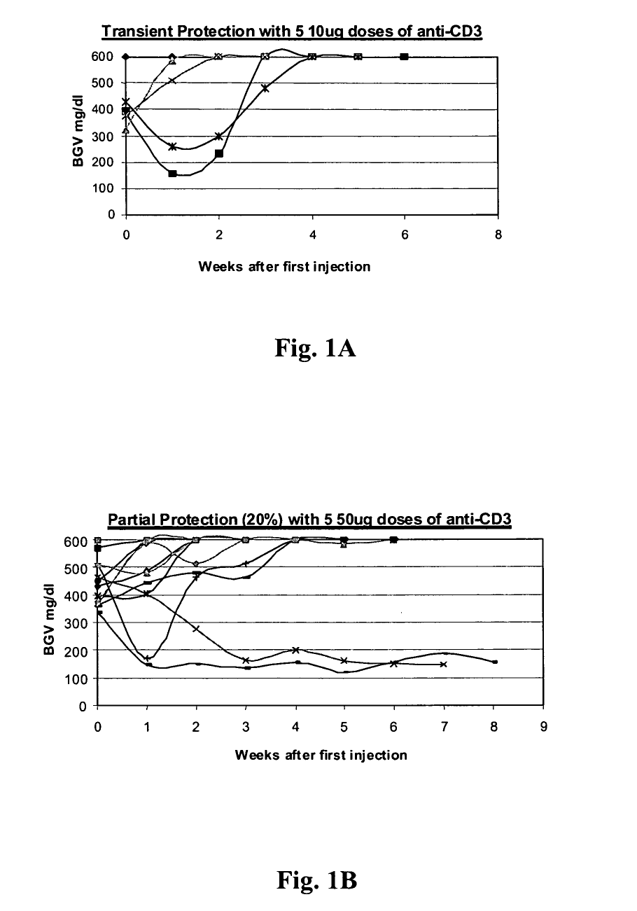

The experience in human studies has been that there is retention or even improvement in insulin responses in 71% of patients at 1 year but there is overall a loss of insulin secretory capacity over the second year. Therefore, in order to study the effects of combined immunotherapy in a model that would have relevance to the clinical situation, the anti-CD3 treatment aspect of the combined therapy has been modified in comparison to prior studies using anti-CD3 treatment alone. In relation to anti-CD3 treatment alone, 50% of the NOD mice treated with a deliberately suboptimal dose of anti-CD3 F(ab′)2 alone (5 times 100 μg i.v.) after recent onset of diabetes are protected (FIG. 1C; see FIG. 1 description above). However, in the NOD model, anti-CD3 F(ab′)2 has to be administered during a therapeutic window (blood glucose values between 250 and 500 mg/dl) to revert T1D. Indeed, mice with blood glucose levels higher than 500 mg/dl are almost never protected after anti-CD3 treatment.

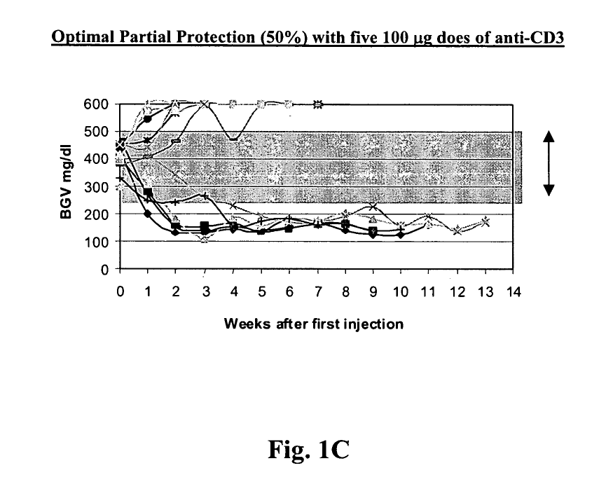

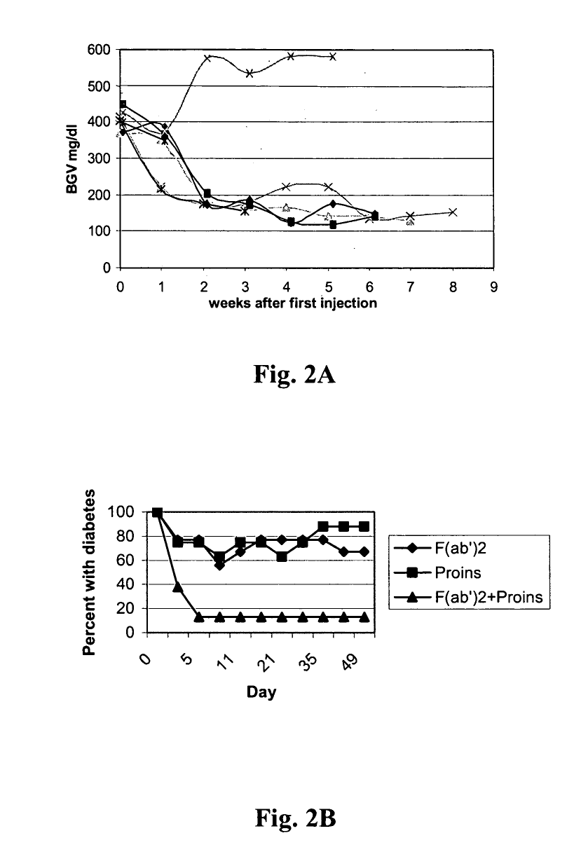

By combining anti-CD3 F(ab′)2 and antigen-specific therapy (here recombinant human GAD65 [rhGAD65] and mouse proinsulin II peptide without the CTL epitope (43)), a synergistic effect was obtained as the number of protected mice was significantly increased over anti-CD3 treatment alone (FIG. 2A). After co-administration of anti-CD3 F(ab′)2 and rhGAD65, 33% of NOD mice remain diabetic while, by using mouse proinsulin II peptide, 100% of the mice are protected 2 weeks after the first anti-CD3 injection. Thus, treatment with anti-CD3 and intranasal proinsulin resulted in 100% protection compared to 50% protection seen with anti-CD3 alone (FIG. 1C) and no protection with proinsulin alone given to recent-onset diabetic NODs (not shown).

Additionally, FIG. 2B shows enhanced remission of diabetes when anti-CD3 mAb is combined with antigen. Female NOD mice, found to have diabetes (diagnosed when the glucose level was >200 mg/dl) were treated with F(ab′)2 fragments of anti-CD3 mAb (145-2C11, n=9), proinsulin peptide (Auspep, n=8), or both F(ab′)2 fragments of anti-CD3 mAb and proinsulin peptide (n=8). The dose of proinsulin peptide used was 40 ug i.n. on days 0, 1, 7, 12 and the dose of the F(ab′)2 fragments of anti-CD3 mAb used was 50 ug i.v. on days 0-4. The blood sugar levels were measured using a hand held glucose meter 2-3×/week daily for 7 weeks. Mice that were found to have a glucose level>200 mg/dl were scored as having diabetes. Treatment with the combination of the F(ab′)2 fragments of anti-CD3 mAb with proinsulin peptide showed significant improvement in the rates of diabetes at day 42 in mice treated with both reagents compared to those treated with the F(ab′)2 anti-CD3 mAb (p<0.05) or proinsulin (p=0.01) alone.

These results clearly indicate the synergistic protective effect against autoimmunity provided by coadministration of anti-CD3 and self-antigen as compared to the administration of anti-CD3 or self-antigen alone.

RIP-LCMV model (RIP-GP mice):

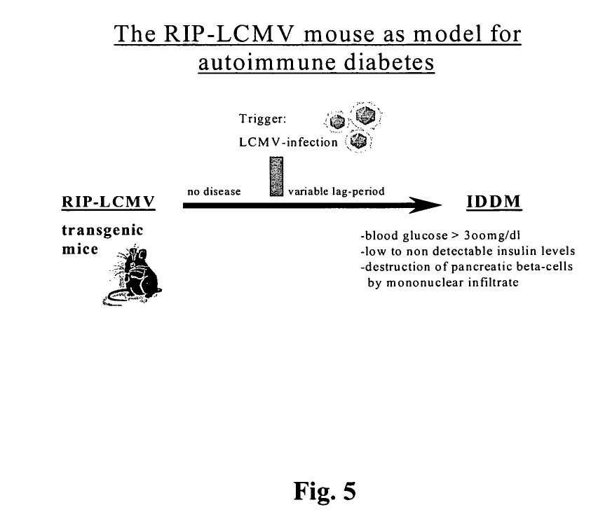

RIP-LCMV transgenic mice can be used as a mouse model for virally induced type 1 diabetes. The mice express a well-defined target autoantigen exclusively in pancreatic β-cells but not any other organs (see FIG. 5). Using the rat insulin promoter (RIP), this approach results in generation of transgenic lines that either express the glycoprotein (GP) or nucleoprotein (NP) of lymphocytic choriomeningitis virus (LCMV) in their islets as a self-antigen. Expression of these viral transgenes do not lead to any β-cell dysfunction and the majority of such transgenic mice (>95%) remain healthy over their life span without any signs of islet dysfunction or infiltration (this applies to the LCMV-GP but not the LCMV-NP strain).

These mice thus constitute an ideal tool to manipulate the immune response to one defined self-antigen and test, which type of autoreactive responses would lead to clinically overt disease. Indeed, most dominant and subdominant T cell (CD4 and CD8) epitopes for LCMV have been mapped for the mouse H-2b and H-2d haplotypes. The immune response has been quantified precisely in many different laboratories and most “tools of the trade” such as tetramers, FACS for intracellular cytokine production and ELISPOT assays have been established. Consequently, this experimental model is unique in that the autoreactive immune response can be tracked, manipulated and defined and the initiating self-antigen is known. RIP-LCMV mice have since validated the concept that peripheral tolerance/unresponsiveness to a defined self-antigen (transgene) can be broken by a systemic viral infection leading to attack of β-cells and their eventual destruction. For the “success” of the autoreactive aggressive response, a sufficiently high amount of autoreactive lymphocytes needs to be generated systemically.

Further, our data indicate that activation of antigen presenting cells (APCs) in the islets and maybe, similarly, direct viral infection of the pancreas is usually required for such autoreactive T cells to become pathogenic. Spontaneous disease in RIP-LCMV-NP mice has only recently been discovered (see preliminary data) and has not been investigated in previous studies, because it only develops to a significant degree after 6 months of age. To this date, by the use of genetically deficient (‘knock-out’) mice, perforin, interferon-γ, as well as TNF-α have all been determined as essential for diabetes development in these mice. Systemic effects could not be separated from localized pancreatic/islet specific processes in these studies. RIP-LCMV mice mirror many aspects of human diabetes such as spreading of the autoimmune process to other self (islet) antigen preceding onset of clinical disease and generation of islet-antibodies. These features make it a suitable and highly relevant model for understanding the pathogenesis of human diabetes.

Mice inoculated with LCMV. Virus (LCMV) infection induces type 1 diabetes in RIP-NP/GP mice. The key advantage of this experimental strategy is the defined pathogenesis and availability of multiple analytical methodology reagents. Control experiments include studies in C57BL6 (DbKb haplotype) and Balb mice (LdKd haplotype) that share MHC haplotypes with NOD and NOD-NP mice (DbKd haplotype). RIP-NP or NOD-NP studies for virus-induced diabetes and immune regulation: due to a) higher incidence of diabetes and b) faster diabetes onset kinetics, experimental groups will be smaller and more experiments can be conducted per year. 15 mice per experimental group, 6-8 experimental groups, 4-5 experiments per year.

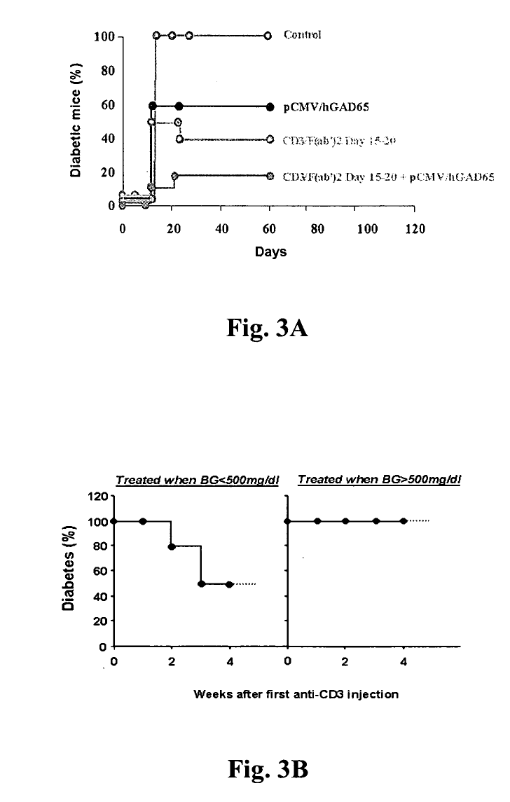

As shown FIG. 3 (see Figure description), after treatment with the non-mitogenic anti-CD3 F(ab′)2 (days 15 to 20 post LCMV infection) approximately 40% of the RIP-LCMV-GP mice are protected. Treatment with hGAD65 (by using an eukaryotic expression vector, pCMV) alone resulted in partial reversion from diabetes (40% protected mice); similar results were previously obtained when porcine insulin B expressing plasmid (pCMV/insB) was used alone to treat RIP-LCMV-NP mice (Coon et al., J Clin Invest (1999) 104:189-94). Here, the combination of anti-CD3 and pCMV/hGAD65 treatments shows a strong synergistic effect in the RIP-LCMV-GP model (80% of the mice were protected).

As observed with the NOD mice (FIGS. 1 and 2), the synergistic effect between anti-CD3 and antigen-specific treatment was only observed when the first anti-CD3 F(ab′)2 injection was given within a therapeutic window (blood glucose [BG] values between 250 and 500 mg/dl) (FIG. 3). Interestingly, if the mice were treated when BG exceeded 500 mg/dl (outside the therapeutic window), they remained diabetic for the rest of their life; in contrast, when anti-CD3 is injected when BG values are lower than 250 mg/dl during the pre-diabetic phase, the mice never turn diabetic (which would be the equivalent to a diabetes prevention but not recent onset trial). This conclusion raises at least two major open questions: (1) what is the best marker (or combination of markers) in humans to define such therapeutic window, and (2) when does such a window occur during the disease course. For the present invention and without being bound by theory, it is contemplated that recent-onset diabetic patients may be the best initial target group for this combinatorial intervention. To extend these studies, the RIP-LCMV-NP model can be used to test the synergistic potential between anti-CD3 and insulin (as peptides or DNA vaccine).

The combination of anti-CD3 systemic therapy with antigen-specific immunization can exhibit a strong synergistic effect in treating recent onset Type 1 diabetes (T1D) in NOD and RIP-LCMV mouse models.

After several years of antigen-specific interventions to treat T1D, the general conclusion is that such treatments have to be given early (during the pre-diabetic phase) in animal models. These interventions might be effective in a prevention trial but not a recent-onset trial (which is a safer time frame for initial human therapy). These studies tested that FcR non-binding anti-CD3 mAb alters the immune response to diabetes antigens that are administered together with the antibody. Furthermore, aggravations of autoimmunity and anaphylactic reactions have been seen after administration of altered peptide ligands in humans in MS (but not diabetes trials) (Neurocrine, published information and personal communication; Bielekova et al., Nat Med (2000) 6:1167-75; Kappos et al., Nat Med (2000) 6:1176-82;) as well as insulin B chain peptides in mouse models (Liu al., J Clin Invest (2002) 110:1021-7; Pedotti et al., BMC Immunol (2003) 4:2. Without being bound by theory, it is believed that the immune modulation caused by anti-CD3 mAb will avoid autoaggressive responses and in this way, will increase efficacy and safety. Therefore in these studies efficacy and various treatment parameters are tested to establish the utility, optimal timing of treatment, and safety of anti-CD3 mAb with antigen.

Methodology and Experimental Plans:

Animal models of T1DM: Preclinincal experiments can use two animal models, for example, for type 1 diabetes, the RIP-LCMV model for virally induced autoimmune diabetes and the NOD (non-obese diabetic) mouse model for spontaneous disease. The intention is to compare the therapeutic efficacy in two different model systems in order to find similarities and discrepancies and get a good impression as to the robustness of the combinatorial therapy. This is particularly important, since human pre-diabetics are genetically heterogeneous and might have different underlying immunological causes for their beta-cell destruction and autoaggressive response. An additional reason for using the viral antigen models is that they offer the distinct advantage that auto-aggressive lymphocytes can be easily tracked (which can be difficult for spontaneous autoimmune responses in the NOD) and the time point for initiating the autoimmune reaction can be experimentally chosen (allowing for a clear anticipation and comparison of non-diabetic, pre-diabetic and recently diabetic stages). Furthermore, RIP-LCMV mice exhibit many features of type 1 diabetes, such as involvement of autoreactive CD4 and CD8 lymphocytes, APC activation, auto-antibodies to insulin and GAD preceding clinical disease and dependence on genetic factors.

Mice and reagents: RIP-LCMV (age 6-10 weeks that usually develop T1D within 11-16 days post LCMV infection [105 pfu i.p.]) and NOD mice can be treated with IgG2a anti-CD3 (Ala-Ala) at days 0, 1, 2, 3 and 4 post onset of hyperglycemia (Blood Glucose>250 mg/dl). In preliminary studies using F(ab′)2 anti-CD3 mAb, which has very similar properties, the invention has shown that 100 μg/injection (5 consecutive days after recent onset diabetes) protects approximately 50% of the RIP-LCMV or NOD mice treated (FIG. 1C). Similar studies can be done with this form of the anti-CD3 mAb (IgG2a anti-CD3 (Ala-Ala)) and other forms to duplicate these findings. Without being bound by theory, the invention provides that anti-CD3 treatment ‘opens a window’ for antigen-specific therapy in the remaining non-protected mice (˜50%), which then would increase the number of protected mice (this is supported by the results in FIG. 3). This is an optimal baseline response rate to study the combined approach because any small differences caused by the combination will be clearly apparent. Furthermore the experimental system has relevance to the clinical situation at 2 years after mAb treatment in which 24% of drug treated patients had a MMTT response that was 80% of baseline (vs. 11% in the controls).

Mice (8-10 per group) can be divided into 14 different groups, for example, to receive (i) anti-CD3 alone, (ii) anti-specific therapy alone or (iii) anti-CD3 in combination with antigen-specific therapy.

Group 1: Anti-CD3 mAb alone (10 μg i.v.) days 0, 1, 2, 3, and 4 post onset of hyperglycemia.

Group 2: Anti-CD3 mAb with antigen (see list below) started together with anti-CD3 mAb after the onset of hyperglycemia. These studies test the effects of administering the anti-CD3 mAb with the antigen. Following administration of anti-CD3 mAb there is a reduction in the number of circulating T cells, possibly reflecting egress of the activated T cells from the vascular compartment but not likely reflecting apoptosis of all T cells. Therefore, although reduced in number, the T cells that remain may be instrumental in modifying the response to antigen administered at the time of the anti-CD3 mAb.

Group 3: Anti-CD3 mAb with antigen started 10 days after anti-CD3 mAb. In these mice, there will be recovery of T cells following treatment with the anti-CD3 mAb. This group tests the lasting effects of the anti-CD3 mAb treatment

Group 4: Antigen alone (see list below) started directly after the onset of hyperglycemia.

Group 5: Antigen alone (see list below) started 10 days after the onset of hyperglycemia.

Group 6: Untreated (PBS-injected) NOD or RIP-LCMV

As control for the antigen-specific treatment, pCMV only and irrelevant peptide or protein only can be used. Blood glucose can be assessed twice a week during the pre-diabetic phase and the first week of treatment, and weekly after.

Anti-CD3 Antibodies: 3 different sources of anti-CD3 can be tested, for example, in order to detect differences and parallels. This is important for transferring findings in the mouse to the human situation, in which anti-CD3-Ala-Ala is being used.

Anti-CD3-Ala-Ala: This antibody exhibits similar functions in the mouse compared to humans and has a mutated Fc-binding region. It may be non-mitogenic, but induces signaling in T cells.

Anti-CD3-IgG3: Similar to the Ala-Ala version, as this antibody exhibits similar functions in the mouse compared to humans and has a mutated Fc-binding region. It is non-mitogenic but induces signaling in T cells.

Anti-CD3 Fab′2 (commercially available from BioSource)—this antibody is derived from mouse 2C11 and is FcR non-binding—It is non mitogenic but induces signaling in T cells.

Antigenic regimens to be evaluated in synergy with anti-CD3 mAb: One can test a variety of islet autoantigens that have been shown to induce regulatory cells and prevented diabetes in animal models. Various routes of administration can be tested as well. Ultimately one goal of the invention is to identify candidates of the list below that exhibit optimal synergy with anti-CD3 and then focus on that antigen for clinical trials (see Example 3).

Intranasal human insulin and human insulin analog X38 (0.05 mg/dose in 110 μl PBS)-days 0, 3, 7, 12. Obtained from NovoNordisk. Of particular interest is their insulin analog X38 that has a 1000-fold lower insulin-receptor binding capacity.

Porcine insulin-B chain (5 mg/kg s.c. in 100 μl)-Days 0, 3, 7, 10, 15. Obtained commercially from Novo Nordisk, Bagsvaerd, D K (Liu et al., J. Clin. Invest., (2002) 110:1021-7). A modified peptide has been produced that has slower absorption and does not induce the anaphylaxis that has been seen with insulin B9-23 peptide alone. This modified peptide can be tested as well.

Porcine insulin-B chain APL (altered peptide ligand) (5 mg/kg s.c. in 100 μl)-days, 3, 7, 10, 15 is obtained commercially from Neurocrine, San Diego, Calif., USA, (Alleva et al., Diabetes (2002) 51:2126-34).

Human GAD 65 protein (hGAD65) (100 μg s.c. in 300 μl PBS, obtained commercially from Diamyd, Stockholm, Sweden) days 0, 1, 7, 12.

Mouse proinsulin II peptide with and without CTL epitope (proinsulin peptide) alone (40 μg intranasally in 10 μl PBS)-days 0, 3, 7, 12 is obtained from Auspep, Melbourne, Australia, 43.

DNA vaccines based on pCMV vector expressing porcine insulin B chain or human GAD: (100 mg i.m. in 100 ml PBS)— administration is, days 0, 3, 7, 12. These are produced under endotoxin-free conditions. Regulatory cells can be induced with such DNA vaccines. (Coon et al., J. Clin. Invest. (1999) 104:189-94).

HSP60 peptide: It has been suggested that HSP60 is an important antigen in human and murine diabetes. Vaccination with HSP60 was shown to prevent autoimmune diabetes induced with multiple doses of streptozotocin and in the NOD mouse (Elias et al., Proc Natl Acad Sci USA (1991) 88:3088-91.54; Elias et al., Diabetes (1994) 43:992-8; Birk et al., J Autoimmun (1996) 9:159-66). Indeed, a trial in patients with recent onset disease has already shown clinical efficacy (Raz et al., Lancet (2001) 358:1749-53).

All antigens were chosen based on their previous proven efficacy in animal models (when given during the prediabetic phase) and the fact that they are in or are being considered for antigen-specific trials in patients with T1DM. As noted above antigens can be given either after the onset of hyperglycemia or following a 10 day delay. The effects of treatment on reversal of diabetes can be studied by measurement of glucose levels and by evaluation of insulitis. In addition, real time PCR can be performed in islets isolated from mice in the various groups to determine whether the treatment regimen has altered the expression of cytokines in the islets.

Thus, a strong synergistic effect of anti-CD3 and islet specific-antigen administration is expected and an optimal antigenic formulation can be identified by using the NOD and RIP-LCMV mice models in combination.

The results show that the concept of ‘therapeutic window’ may be important to achieve therapeutic efficiency for T1D (FIG. 1C). While the NOD model shows a relatively ‘long’ therapeutic window (due to the slower disease course), such window in the RIP-LCMV model is shorter and more difficult to determine (rapid and strong pancreatic destruction after LCMV infection). One possibility is to widen this window in the RIP-LCMV model by using lesser virus quantities for infection or by infecting mice at 8-12 weeks and not 6 weeks after birth (both strategies should delay T1D and decrease the pancreatic aggression and open a larger therapeutic window if necessary).

Thus, the above experiments can identify the optimal antigen-anti-CD3 combination and timing and ensure that no acceleration of beta cell destruction will occur in two distinct animal models. From the data, proinsulin appears to be the most promising candidate, which might also be a good choice, since recently tracking of pro-insulin specific responses has been reported in humans showing TH2-like responses in normal versus Th1-like responses in pre-diabetic individuals. Indeed, being able to track antigen specific responses would provide additional guidelines for the clinical trial (see Example 3).

The Table below provides a list of where some of the anti-CD3 and antigen reagents used in the Examples can be obtained:

TABLE 2
Exemplary Sources of Antibodies and Antigen Reagents

| | | Reference |
|---|---|---|
| Antibody/Antigen | Source | (hereby incorporated) |
| Anti-CD3 F(ab′)₂ | BioExpress, New | Chatenoud et al., Lancet |
| | | (1989) 2: 164; |
| clone 2D11 | Hampshire, USA | Chatenoud et al., C.R. |
| | | Acad. Sci. III (1992) |
| | | 315: 225-8. 1992; |
| | | Chatenoud et al., J. |
| | | Immunol. (1997) 158: |
| | | 2947-54; |
| | | von Herrath et al., J. |
| | | Immunol. (2002) 168: |
| | | 933-41. |
| Anti-CD3 | J.A. Bluestone, San | Herold et al., J. Clin. |
| (hOKT3gamma1[Ala- | Francisco, CA, | Invest. (2003) 111: |
| Ala]) | USA | 409-18. |
| | | Herold et al., N. Engl. J. |
| | | Med. (2002) 346: |
| | | 1692-8. |
| Ins B 9-23 | Eli Lilly | Homann et al., J. |
| | | Immunol. (1999) 163: |
| | | 1833-8. |
| Ins B 9-23 APL | Neurocrine, San | Alleva. et al., Diabetes |
| | Diego, CA, USA | (2002) 51: 2126-34. |
| Ins B hypoallergenic | G. Eisenbarth, | Liu et al., J. Clin. Invest. |
| | Denver, CO, USA | (2002) 110: 1027-7. |
| Proinsulin peptide | Auspep, | Martinez et al., J. Clin. |
| (without CTL epitope) | Melbourne, | Invest. (2003) 111: |
| | Australia | 1365-71. |
| | (L. C. Harrison | |
| | group) | |
| pCMV/insB | Disclosed herein | Coon et al., J. Clin. |
| | | Invest. (1999) 104: |
| | | 189-94. |
| pCMV/hGAD65 | Disclosed herein | N/A |
| rhGAD65 | Diamyd, | N/A |
| | Stockholm, Sweden | |

### Example 2

### T Regulatory Cells Induced by Anti-CD3 and Self-Antigen can be Used to Treat Autoimmunity

The data in Example 1 shows that the combined administration of anti-CD3 and autoantigen can have a synergistic effect in inducing tolerance and protecting against autoimmunity. In building upon these results, the present invention includes methods where T regulatory cells that have been exposed to autoantigens and anti-CD3 treatment can be isolated and/or expanded in vitro in order to use the cells themselves for autoimmune therapies. Prior to clinical testing in humans, preclinical testing can be conducted to determine whether any particular combinatorial treatment (i.e., any particular form of anti-CD3 in combination with any particular self-antigen) induces regulatory T cells (Tregs) and/or systemic immune deviation.

In preliminary adoptive transfer studies (see Table 3 below), using lymphocytes from anti-CD3 treated or anti-CD3+ Ag treated RIP-LCMV donors, only CD4+ cells from anti-CD3 and antigen treated RIP-LCMV mice were capable of protecting syngeneic, pre-diabetic non-immune suppressed hosts from development of diabetes. Cells from mice that were treated with anti-CD3 alone were not able to mediate protection. This study shows that induction of T regulatory cells after antigen administration occurs efficaciously in anti-CD3 and antigen treated mice. In contrast to earlier observations by Chatenoud, who used immunodeficient recipients to detect T regulatory activity in lymphocytes from anti-CD3 treated NOD donor mice, such regulation in immune competent mice may depend on more robust T regulatory induction.

Induction of T regulatory cells with anti-CD3 mAb and antigen: The in vitro expansion of antigen-specific T regulatory cells and their re-introduction in type 1 diabetes models can prevent disease. The results from previous published and unpublished studies are summarized in the following table. In brief, T regulatory cells isolated from NOD mice or RIP-LCMV mice that had received islet antigen specific immunizations in conjunction with or without anti-CD3 were stimulated in vitro in the presence of IL-2 (and IL-4 in some studies) and, in some cases as indicated in the presence of insulin B chain (RIP-LCMV studies) or beads bearing I-A^{g7} tetramers presenting the BFDC2.5 or GAD peptides. Following in vitro expansion, these cell lines were re-introduced into syngeneic pre-diabetic RIP-LCMV or NOD recipients and development of type 1 diabetes was monitored.

TABLE 3
Summary of Studies of T Regulatory Cells

| | | Recipient % T1DM (wks |
|---|---|---|
| Donor and cell type | In vitro stimulation | of age or after LCMV) |
| RIP-LCMV* | | |
| RIP-LCMV, no Ag | IL-2, IL-4, insB 2-wk | RIP-LCMV; 90-100%, (2 |
| | | weeks) |
| RIP-LCMV, oral | IL-2, IL-4, insB 2-wk | RIP-LCMV; 40-55%, (2 |
| insulin | | weeks) Homann et al, |
| | | Immunity (1999) 11: |
| | | 463-72 |
| RIP-LCMV, | no stimulation | RIP-LCMV; 40% (2 weeks) |
| ins-CTB | | Asseman et al., Novartis |
| | | Found Symp (2003) 252: |
| | | 239-53; discussion |
| | | 253-67 |
| RIP-LCMV, pinsB | no stimulation | RIP-LCMV; 50% (2 weeks) |
| | | Coon et al., J Clin Invest |
| | | (1999) 104: 189-94 |
| RIP-LCMV, | no stimulation | RIP-LCMV; 50% (2 weeks) |
| pinsB + antiCD3 | | |
| RIP-LCMV, | no stimulation | RIP-LCMV; 95% (2 weeks) |
| anti-CD3 alone | | |
| Balb/c CD25+ | IL-2 LCMV-Ag 2-wk | RIP-LCMV; Reduction of |
| | | LCMV-CD8 CTL by 50% |
| No cells | none | NOD RAG−/−; 100% |
| | | (3-45 days) |
| NOD, CD25+/ | IL-2, 3 weeks | NOD RAG−/−; 75% |
| CD62L | | (50d post transfer) |
| NOD BDC 2.5 | IL-2, 3 weeks | NOD RAG−/−; 0% |
| CD25+ | | (100d post transfer) |
| No cells | none | NOD CD28−/−; 100% |
| | | (at 10 weeks of age) |
| NOD BDC 2.5 | IL-2, 3 weeks | NOD CD28−/−; |
| CD25+ | | 0% (age 22 weeks) |

*Note that without antigenic stimulation in vitro, more cells need to be transferred for protection (without stimulation 5 × 10⁶ cells required) compared to antigenic stimulation (requires only 2 × 105 cells).

The following studies describe how to track T regulatory cells by the products they produce as well as by directly identifying the cells using tetramers and ELISPOTs.

In particular, T regulatory cells can be tracked by the cytokines they produce, for example, preliminary results indicate that T regulatory cells have a cytokine production profile that may comprise IL-10 and IL-4, or IL-10, IL-4, IFN-γ and TGF-β. Specifically, IL-10 may be a key cytokine produced by T-regulatory cells. Additionally, TGF-β might also be very important (5). In both cases, in vitro stimulation can enhance T regulatory function and the presence of IL-2 appears crucial.

Methods

Assessment of systemic immune deviation after anti-CD3 (+/−antigen): Systemic cytokine levels in serum can be assessed by ELISA. Cytokine production that occurs in a polyclonal fashion can be assessed directly ex vivo by exposing sorted lymphocyte populations (from pancreas, PDLN, spleen and PBMCs) in ELISPOT assays and, in addition, after polyclonal in vitro stimulation (anti-CD3/CD28 and SEB). When these studies are first conducted in mice, they allow one to draw parallels to human clinical investigations in respect to systemic cytokine shifts observed after anti-CD3.

Tracking and analysis of regulatory T cells: Regulatory T cells from protected mice can be tracked, analyzed, transferred into recipient mice and finally general immune deviation and cytokine profiles after combinatorial therapy can be compared to single therapies. The following techniques can be used to track and analyze the regulatory T cells. For identification of T regulatory cells in donors and recipients, the invention can use antigen specificity, cytokine production as well as expression of CD25+, FoxP3 and GITR as markers using a combination of flow cytometry and western blot analysis. Not all of these are suited to sort cells prior to transfer (see below), but nevertheless they will be very useful to assess systemic changes in profiles of T regulatory cells.

Adoptive transfers to assess presence of regulatory lymphocytes: The fraction of lymphocytes from a given organ that has in vivo autoimmune suppressive activity can be assessed by adoptive transfer and sorting as already described (Homann et al., Immunity (2002) 16:403-15; Homann et al., Immunity (1999) 11:463-72). In brief, adoptive transfer of CD4 lymphocytes has resulted in prevention of disease in non-immune suppressed, un-manipulated (non-irradiated) pre-diabetic syngeneic recipients. This is a realistic, quantitative and reliable way to test function and existence of regulatory T cells in vivo because homeostatic effects and cell expansion in an immunologically ‘empty’ environment dose not occur. Importantly, sorting after phenotyping right before transfer can identify the crucial subpopulations.

The following phenotypes can be used for sorting T regulatory cells: (a) Cell surface markers in transfers: CD25+, CD4+, CD62L+ (MACS sort) or (b) Cytokines needed by T regulatory cells: IL-2, 4, 10, IFN-γ (Miltenyi beads sort).

Further, in vitro culture enables amplification of antigen specific subsets. Adoptive transfer can be performed with the RIP-LCMV and NOD models. Splenocytes and pancreatic draining lymph node (PDLN) from protected mice (anti-CD3 or islet antigens treated alone or in combination) can be used to collect the putative regulatory cells. Initially, a pool of splenocytes or PDLN cells can be used to transfer into non-irradiated syngeneic recipients (intraperitoneal or intravenous injection in 6- to 8-week-old mice for the NOD model, and day 5-6 after LCMV infection for the RIP-LCMV model). In a second set of experiments, the potential regulatory T cells can be tracked with specific purifications in the T cell population (purification according to the expression of CD4, CD25 and CD62 cell surface markers, for example, and by using magnetic beads or FACS-Vantage technology). These sub-populations can be injected into recipients as described above to assess the presence of T regulatory cells on autoimmunity.

Tracking of antigen specific aggressive and regulatory T cells in lymphoid organs and PBMCs: The following approaches can be used to detect LCMV NP-, GP- and GAD, proinsulin and insB-specific CD4 and CD8 lymphocytes in the RIP-LCMV mice or in the NOD model. The regulatory T cells can be tracked in these sub-populations by using several sources of T cells (islets and pancreas infiltrating lymphocytes, splenocytes, and lymph node cells).

a) Peptides: Epitopes derived from the LCMV-NP transgene are the dominant Db-restricted NP396-404 [amino acids FQPQNGQFI (SEQ ID NO:1)] and the subdominant Kb NP314-322 [(W)PIACRSTI (SEQ ID NO:2)]. Other viral epitopes recognized by NP CD8+ T cells are Db GP33-41 [KAVYNFATC(SEQ ID NO:3)], Db GP276-286 [SGVENPGGYCL (SEQ ID NO:4)] and Kd GP283-291 [GYCLTKWMI (SEQ ID NO:5)] and are used as controls. Furthermore, the Kd INS-B15-23 [LYLVCGERG (SEQ ID NO:6)], the mimotope Kd NRP-A7 [KYNKANAFL (SEQ ID NO:7)] as well as the I-Ag7-restricted INS-B9-23 [SHLVEALYLVCGERG (SEQ ID NO:8)] and Insulin C13-A5 as well as GAD protein from Diamyd can also be used. These peptides can also be utilized in ELISPOT and proliferation assays. Cytokines that can be measured are IFN-γ, IL-10, TNF-α, IL-4 and IL-10 (see ELISPOT section).

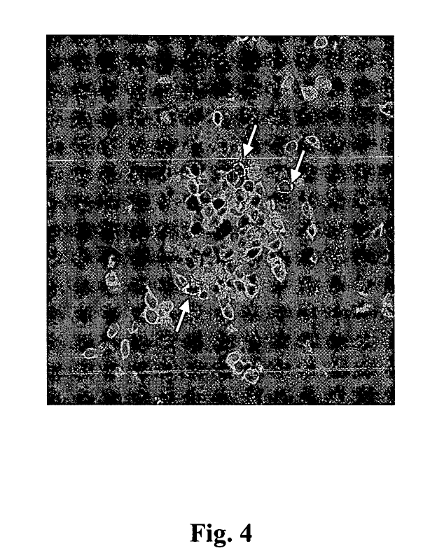

In situ tetramer stains: This is the least invasive approach that will allow detecting CD8 cells directly ex vivo on tissue sections. This approach is established, and an example is provided in FIG. 4. For identification of β-cell antigen-specific CD8+ T cells by flow cytometry or in situ staining, the following phycoerythrin-(PE) or allophycocyanin-(APC) conjugated tetramers can be used: DbNP396-404, KdINS-B 15-23 and KdNRP-A7 (mimotope). Tetramers that recognize LCMV GP-specific epitopes (DbGP33-41 and KdGP283-291) can serve as controls.

b) Tetramers and FACS intracellular cytokine staining: Tetramers for both MHC class I or II molecules are available. Multicolor flow cytometric analyses are performed using single cell suspensions from different origins (PBL, splenocytes, draining lymph node, etc.) as described. After restimulation in vitro, cells can be stained for selected surface markers as CD8, CD4, CD25, CD44, CD62L, CD69, etc. or antigen-specific TCR by using tetramers technology (described above). Then, followed by fixation and permeabilization and intracellular staining for IFN-γ, TNF-α or IL-4. For selected experiments, stains with up to 7 different colors can be used on a FACS Vantage.

c) ELISPOT assays: Antigen specific stimulation is required to detect cytokine production (INF-γ, IL-4, IL-10, and IL-5). However, the strength of this approach lies within the fact that only very few cells are needed to obtain reliable results. This technique has been conducted with good success (von Herrath, et al., J. Immunol., (2002), 168:933-41; Coon, et al., J. Clin. Invest., (1999), 104:189-94).

d) Proliferation and ELISA for cytokines in supernatants: This strategy requires the most cells and most extensive in vitro stimulation. For quantification of secreted cytokines, single cell suspensions of organs harvested are cultured in the presence of peptides and supernatant is collected 48-72 hours later and stored at −20° C. for analysis of IFN-γ, IL-2, IL-4, IL-10 and TGF-β.

The above techniques allow one to detect and characterize, in terms of cell surface markers or cytokine expression, T regulatory cells induced during different treatments of anti-CD3 and antigen (for example, anti-CD3 F(ab′)2 or islet antigens treated alone or in combination). Indeed, no information is yet available concerning the phenotype of such T regulatory cells induced by a combined treatment anti-CD3/antigen in comparison with anti-CD3 or antigen-specific treatment alone. It is very important to note that tracking of antigen specific cytokine producing cells by ELISPOTS or, where available, by direct MHC class II tetramer staining, can be instrumental to guide one to perform similar tracking during the human trials. Since adoptive transfers are not possible in humans, the precise cytokine profiling with or without antigenic exposure ex vivo may be essential. Without being bound by theory, the present invention provides that anti-CD3 alone will lead to systemic cytokine shifts, and combination with antigen will lead to generation of antigen specific T regulatory cells that secrete cytokines such as IL-10, IL-4 and IL-13. Similar cytokine assessments in patients receiving this combinatorial immunotherapy can be used to monitor/assess/validate the effectiveness of treatment.

One technical problem that may arise is that T regulatory cells are found in a low proportion in the T cell population. However, the use of sensitive techniques such as ELISPOT, ELISA, and FACS analysis, should provide reliable and consistent results. For example, prior adoptive transfer experiments show that the T regulatory cells can be successfully isolated, tracked and analyzed (Homann, et al., Immunity, (1999), 11: 463-72).

### Example 3

### Coadministration with Modified Anti-CD3 Antibodies

The data from Example 1 shows that combination of anti-CD3 F(ab′)2 systemic therapy with an antigen-specific approach exhibits a clear synergistic effect in treating recent onset T1D. Several autoantigens (DNA vaccine, full protein or peptides) are under therapeutic evaluation in order to determine the best combination giving a maximal protection. Therefore, the model systems for T1D (Examples 1 and 2) can be used to test new reagents for use that mimic the effects that are seen in patients with T1DM treated with anti-CD3 mAb hOKT3γ1(Ala-Ala). The proposed studies in Example 4 pertain to the design of clinical trials in which to test the alterations of immune responses by the combination of antigen with anti-CD3 mAb, provide information on the safety of the combination, and also develop an understanding of the mechanisms involved.

### Example 4

### Clinical Trial Design for the Treatment of Diabetes by the Coadministration of Anti-CD3 Antibodies and Autoantigens

The following are four specific non-limiting milestones contemplated to be achieved by preclinical and clinical trials:

(1) Reproducibility of preclinical synergy data at 2 centers: It is important that recent onset-diabetes and not only pre-diabetic mice are protected. There are too many immune based interventions that protect NOD mice when given during the pre-diabetic phase, but only very few that can re-establish euglycemia in recent-onset NODs. In Example 1 (FIG. 2), the dosing techniques for administration of the anti-CD3 mAb was modified so that about 50% of treated mice reverse diabetes at the time of onset in the NOD mouse and the LCMV model. The data from FIG. 1C provided a window in which to evaluate the effect of adding antigen. In addition, if the effect is not sustained (i.e. >3 months), then it can be readily determined whether repeated administration of the antigen reduces the relapse rate. The NOD studies can be independently confirmed at two centers before proceeding with a clinical trial.

(2) Clear antigenic candidate: Based on the studies described above, an antigen can be selected that is optimal for maintaining the reversion rate of diabetes. In case two or more antigens show similar optimal synergy with anti-CD3, the antigen can be chosen as the trial candidate that shows best availability in GMP (good manufacturing practice) formulation. The data herein suggests that Proinsulin and/or GAD65 may fulfill these criteria.

(3) Source for Antigen to be used in the clinical trial: The potentially available antigens for clinical use are listed below in Table 4. In the unlikely event that a new peptide needs to be made, it can be produced by an outside manufacturer under GMP conditions. An IND (investigational new drug) application for use of this peptide can be filed and a small Phase I dosing/safety trial can be carried out in patients with established diabetes.

(4) No severe side effects or detectable augmentation of autoaggressive lymphocytes. It is imperative that the optimal combination of antigen and anti-CD3 does not aggravate disease in either of the two models or sites. Furthermore, allergic reactions that are severe following repeated peptide injections cannot occur. There have been reports in NOD mice of fatal anaphylactic reactions to both insulin peptide and GAD65 (Liu, E. et al., J Clin Invest (2002) 110:1021-7.51; Pedotti et al., BMC Immunol (2003) 4:2). In the case of insulin peptide B9-23, the attachment of 2 arginines to the C-terminus slows an absorption profile which appears to prevent the anaphylaxis that has been observed. This modified peptide can be tested in parallel with insulin B9-23 to determine whether this modification is required to prevent anaphylaxis when the peptide is administered with anti-CD3 mAb. As noted below, Phase I trials with an altered B9-23 insulin peptide have already been done and anaphylaxis was not seen. Likewise anaphylaxis was not reported in the Phase I trials of alum-GAD65. Nonetheless, preclinical studies proposed herein include mice that are administered with more than one dose of antigen to determine whether anaphylaxis is induced and the effect of anti-CD3 mAb on this side effect.

Likewise, while the present methods show protective effects of anti-CD3 mAb on modulating T cell responses to antigens, the combination of antigen with a T cell agonist may augment an undesired reactive response to the antigen or to other cells. This can be studied in the proposed preclinical experiments. In parallel, other early phase studies using the proposed antigens in patients can be ongoing. The data obtained from these studies can be reviewed with particular attention to the effects noted above as well as any long-term side effects. Since the Stiff-man syndrome is associated with autoantibodies against GAD65, the preclinical data and early phase clinical data can be reviewed for any evidence of neurologic events and consider the duration of follow up of subjects in planning the monitoring of the proposed trial.

The clinical translation of the mechanisms of antigen with FcR non-binding anti-CD3 mAb is that insulin production in T1DM can be prevented from loss (and even improved) with FcR non-binding anti-CD3 mAb and maintained by repeated administration of the antigen. This derives its rationale from the understanding of the mechanisms of the combination of these two approaches that is further developed in Examples 1-3 above. These preclinical studies provides a guide in terms of selection of antigen, timing of administration of antigens and anti-CD3 mAb, the markers that can be used to assess the immunologic effects of treatment, and, most importantly, the safety of the combination. The present methods can be used in clinical trials.

The trial is to test the effects of antigen with anti-CD3 mAb on the loss of insulin production in patients with new onset Type 1 diabetes. Treatment with anti-CD3 mAb and antigen can be compared to intensive glucose control and observation. The duration of the trial is 2 years. The trial is also to determine the effects of antigen with anti-CD3 mAb on T cell responses to the therapeutic and other antigens.

B. Background:

Eisenbarth originally described the natural history of T1DM in which there is a progressive linear loss of insulin secretion over time (Eisenbarth, N Engl J Med (1986) 314:1360-8). At some point in this course, clinical disease becomes manifest. Although original morphometric studies suggested that as much as 90% of P cell mass was lost at presentation, our more recent studies in which we measured insulin secretory rates (ISR) in the first 2 years of T1DM indicate that the impairment at presentation is less and that ISR's to a physiologic stimulus (mixed meal tolerance test, MMTT) was 50% of normal (Gepts, Diabetes (1965) 14:619-633; Steele, et al., Diabetes (2004) 53:426-433). Nonetheless, over time, nearly all patients with T1DM, particularly children, lose the ability to make any detectable insulin, although exceptions have been described (Steele, et al., Diabetes (2004) 53:426-433; The DCCT Research Group. J Clin Endocrinol Metab (1987) 65:30-6). Retention of insulin secretion, however, is clearly an important clinical goal because it is associated with improved glycemic control and therefore, likely is to reduce the risk of long term complications if it can be maintained. Faber et al., Diabetologia (1977) 13:263-8; The Diabetes Control and Complications Trial Research Group. N Engl J Med (1993) 329:977-86.

The preliminary data, shown below and in the preceding Examples, supports the notion that T cells exposed to anti-CD3 mAb have regulatory function. However, there were two limitations to use of the anti-CD3 mAb OKT3, including the development of a human anti-murine response to the murine immunoglobulin and a cytokine release syndrome that occurs following the crosslinking of the CD3 molecule and T cell activation in vivo (Chatenoud et al., Transplantation (1990) 49:697-702; Chatenoud et al., Transplant Proc (1990) 22:2605-8; Chatenoud et al., Curr Top Microbiol Immunol (1991) 174:121-34); Chatenoud et al., Transplant Proc (1993) 25:47-51). To circumvent these problems, a humanized anti-CD3 mAb has been developed that has the same CDR region as OKT3 but with a mutation in the Fc portion of the molecule to reduce FcR binding (Xu et al., Cell Immunol (2000); 200:16-26). This molecule, hOKT3γ1 (Ala-Ala) causes binding and modulation of the TCR in a manner similar to OKT3 but does not cause the same T cell activation in vitro or in vivo. More recent studies have suggested that the mAb does in fact deliver an activation signal to T cells, but one that results in relatively greater production of IL-10 instead of IFN-γ which occurs following activation with OKT3 (Herold et al., J Clin Invest (2003) 111:409-18).

A Phase I/II trial of hOKT3γ1 (Ala-Ala) for treatment of patients with new onset Type 1 diabetes was initated in 1999. A total of 42 patients were recruited for this randomized trial—½ were randomized to a single 12 or 14 day course of the anti-CD3 mAb and the other ½ were observed (Herold et al., N Engl J Med (2002) 346:1692-8). All subjects underwent a MMTT every 6 months to assess the effect of mAb treatment on the natural loss of insulin production over the first 2 years of disease. After 1 year, the average C-peptide (a byproduct of insulin production secreted in an equimolar basis as insulin) response to the MMTT was 98±9.8% of the baseline values whereas in the control group it was 54±7.9% of baseline values (p<0.01). Two years after the single course of treatment at study entry, the response was 71±12.1% whereas in the controls it was 25±7.7% of the levels at study entry (p<0.01). Thus, a single course of treatment with anti-CD3 mAb caused a statistical improvement in insulin responses even out to 2 years, but there clearly was a decline in the effect after 1 year. This is perhaps not a surprising finding as lasting alterations of human immune responses (e.g. vaccinations) generally require repeated immunizations. Thus, these findings show that an additional approach is needed to maintain tolerance to the disease, one that is effective, safe, and specific. Unfortunately, the loss of tolerance was not seen in the original studies of anti-CD3 mAb in the NOD mouse—in fact, even in mice that had failed to reverse diabetes following treatment with anti-CD3 mAb, the mice accepted syngeneic islets without redevelopment of disease Chatenoud L, Thervet E, Primo J, Bach J F. Anti-CD3 antibody induces long-term remission of overt autoimmunity in nonobese diabetic mice (Proc Natl Acad Sci USA (1994) 91:123-7). Therefore, the studies described herein were conceived, in part, with the objective to create a model system in which the interactions between anti-CD3 mAb and antigen could be studied.

It is clear that the mechanism for inducing tolerance by anti-CD3 mAb does not simply involve depletion in effector cells. Following treatment of patients with the anti-CD3 mAb there is a transient reduction in the number of circulation T cells but then an increase even with continuous administration of the mAb. By 2 weeks after the last dose of drug, the circulating lymphocyte counts are 100% of the pretreatment levels. The clinical effect that is seen, however, occurs months and even more than 1 year after the drug is given, at a time when there are no discernable changes in the circulating cells.

Thus the strategy that is currently proposed is one in which administration of islet antigen(s) under the umbrella of anti-CD3 mAb results in a response to the antigen characterized by a non-pathogenic phenotype (see Examples 1-3 above). These cells, in turn, might be expected to regulate pathogenic T cells but clearly would maintain a non-pathogenic phenotype. The invention suggests that the response to islet antigen when administered with the anti-CD3 mAb is analogous to the response recently characterized in normal control subjects in which non-pathogenic cytokines such as IL-10 rather than IFN-γ are secreted. The invention postulates that this can result in immune modulation rather than activation of pathogenic effector cells.

C. Preliminary Data:

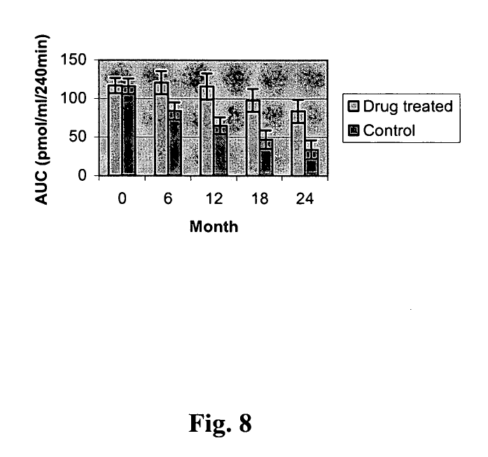

1. Effects of treatment with hOKT3γ1(Ala-Ala) on insulin responses over the first 2 years of T1DM: Patients with new onset (≦6 weeks after diagnosis) were randomized to treatment with a 12 or 14 day course of hOKT3γ1(Ala-Ala)(n=21) or observation (n=19) (see FIG. 8). The area under the curve (AUC) of the C-peptide response to a mixed meal (4 hour mixed meal tolerance test, MMTT) was done every 6 months to evaluate insulin secretory responses. On average, the C-peptide response to the MMTT was 97+/−9.6% of the starting response in the anti-CD3 mAb treated group at 1 year, whereas it was 53+/−7.6% of the starting response in the control group (p=0.001) After 2 years, the responses were 71+/−12% and 25+/−7.7% of the study entry responses respectively (p=0.003). Although the responses declined in the drug treated patients after 1 year, they were significantly greater than in the control group at 2 years.

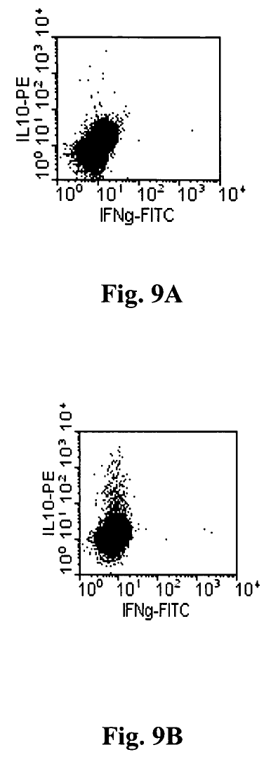

2. Treatment with hOKT3γ1(Ala-Ala) induces cells with a regulatory phenotype in vivo: A subpopulation of CD4+IL-10+T cells in the peripheral blood of patients treated with hOKT3γ1(Ala-Ala) has been identified. PBMC were harvested and frozen from a patient treated with hOKT3γ1(Ala-Ala) at the conclusion of mAb treatment. These cells, as well as those harvested before mAb treatment were thawed and stained for intracellular cytokines, IL-10 and IFN-γ. Markers were placed around surface molecules CD4 and CD3. Cells harvested from patients after anti-CD3 mAb treatment were producing IL-10 in vivo. FIG. 9A (before mAb treatment) and 9B (after mAb treatment) shows the induction of CD4+IL-10+ cells in vivo by treatment with hOKT3γ1(Ala-Ala). These induced cells were IFN-γ negative, predominantly CD45RO+, and generally CCR4+. Thirteen percent of these cells produced TGF-β by surface staining. The spontaneous production of IL-10 in vivo suggests that these cells can exert an immune modulatory effect if present at the time of antigen presentation.

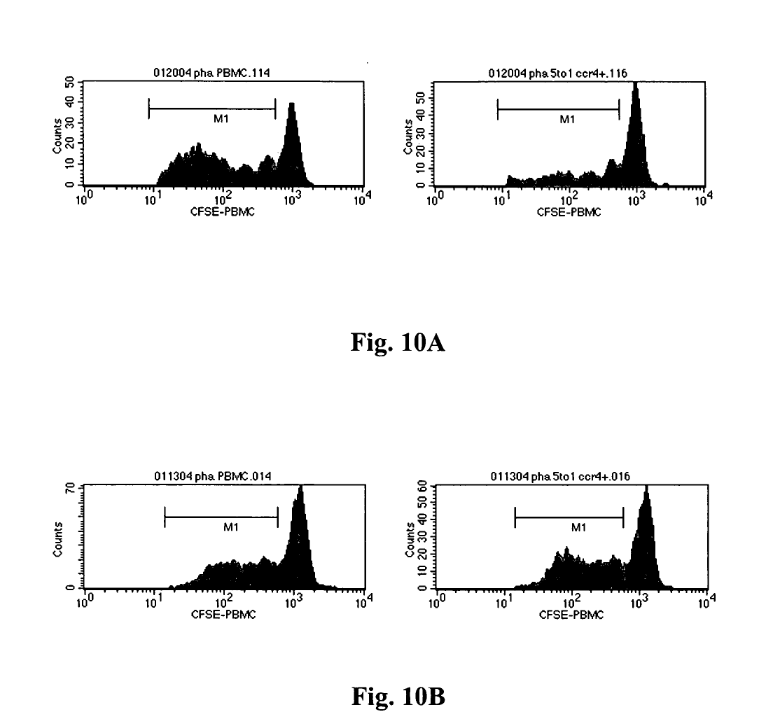

3. Treatment with anti-CD3 mAb induces regulatory T cells in vitro: In order to obtain sufficient numbers of cells for studies of functional properties, anti-CD3 mAb has been utilized to expand regulatory T cells in vitro. PBMC were cultured for 10 days with OKT3 mAb and then CD4+ cells were isolated by sorting. They were then cultured for 19 days with IL-10 and IL-2. These cells were then added to cultures of PBMC from the same individual, stimulated with PHA (phytohemagglutinin). Addition of the cells at a ratio of 1:5 inhibited proliferation of CD4+ T cells as shown in FIG. 10A. The regulatory effect was not a non-specific effect of the cell addition and required anti-CD3 stimulation, because cells cultured for 10 days in IL-10 and IL-2 alone did not inhibit PBMC stimulated with PHA (FIG. 10B). However the culture with IL-10 and IL-2 was necessary for regulation because it did not occur in cells cultured with anti-CD3 mAb alone. The majority (94%) of the CD4+ cells that inhibit PBMC proliferation are CCR4+, 46% are CD62L+, and 93% are CD45RO+. About 5% of the cells are GITR+.

Similar results were obtained by culturing PBMC with hOKT3γ1(Ala-Ala). After 10 days in culture with either hOKT3γ1(Ala-Ala) or IL-10 with IL-2, cells were sorted into CD4+ CCR4+ and CD4+ CCR4− subpopulations based on our findings of IL-10 producing cells ex vivo (4). These cells were then cultured for an additional 2 weeks in IL-10 and IL-2 and then added to PBMC from the same donor and stimulated with PHA. As seen in FIGS. 13A and 13B, CCR4+ cells from cultures with the anti-CD3 mAb inhibited proliferation of the PBMC stimulated with PHA.

In summary, the preliminary data provided in this Example shows that a single course of treatment with anti-CD3 mAb can alter the natural history of T1DM. However, the duration of the effect is not clear at this time and it is likely that an additional form of treatment is needed. Retreatment with the mAb is a possible strategy but the antigen specific approach of the present methods that can be repeatedly administered is preferable. The preclinical studies of the present methods described above in Examples 1-3 with the reagents at hand provide an approach to accomplish the goal, which is to induce tolerance with anti-CD3 mAb and self-antigen and to maintain tolerance by repeatedly administering antigen. The present studies also indicate that anti-CD3 mAb and antigen coadministration may induce antigen-specific regulatory T cells that may be stimulated by repeated exposure to antigen. The clinical efficacy of this approach as well as the mechanisms involved can be studied in this proposed trial.

D. Research Design and Methods:

The effects of the combination of antigen with anti-CD3 mAb on the loss of insulin production in patients with new onset Type 1 diabetes are tested. The proposed clinical trial is for an open label, randomized controlled trial in which treatment with anti-CD3 mAb and antigen is compared to intensive glucose control and observation. The duration of the trial is for 2 years.

Rationale: Preclinical studies (see prior Examples) in the NOD and LCMV models of Type 1 diabetes show that a synergistic effect to prevent diabetes can be induced by combining anti-CD3 mAb with antigen. The clinical research question addressed here is whether immunizing with a diabetes antigen together with anti-CD3 mAb treatment can improve insulin responses for 2 years in patients with new onset T1DM. The studies described in Examples 1-3 can establish the optimal antigen to be used for this trial and provide information regarding the dosing regimen and end points for analysis that can be followed, as well as safety information addressing concerns that have previously been raised. The proposed trial is a 4 arm, open label study of patients with new onset Type 1 diabetes. The primary endpoint is a comparison of the effects of the combination of anti-CD3 with antigen to aggressive diabetes management and observation on C-peptide responses to a mixed meal tolerance test (MMTT) at 2 years after study entry. Secondary endpoints include the effects of treatment on C-peptide responses at 1 year and use of insulin.

Methods:

Research question and study design: An open labeled study is proposed in patients with new onset Type 1 diabetes, to compare the effects of anti-CD3 mAb with antigen with intensive glycemic control and observation, on the loss of C-peptide responses to a MMTT over the first 2 years of T1DM. To study the effects of anti-CD3 mAb or antigen alone on the responses to these antigens, 2 additional study groups are included in which these responses can be studied. These study groups are included primarily for comparison of mechanistic studies to patients in the two primary treatment question groups. However, the study is powered to determine whether these individual treatments have a clinical impact on C-peptide responses compared to intensive glucose control and observation alone. The primary clinical question, however, is the effects of the combination of antibody and antigen compared to intensive insulin control and observation after 2 years.

Possible sources of antigens for clinical use: The studies in the Examples above in the LCMV and NOD mouse show the effectiveness of combining anti-CD3 mAb with either GAD65 or Proinsulin, and other studies implicate an efficacy of insulin peptides alone in the NOD model. In addition, for these suggested antigens, in vitro assays are available that are used to follow the effects of antigen administration with or without anti-CD3 on immunologic responses. A heat shock protein peptide (DiaPep277) has shown beneficial effects in the NOD mouse, multi-dose streptozotocin induced diabetes, and human diabetes, and is tested in combination with anti-CD3 mAb in the studies above (Elias et al., Diabetes (1994) 43:992-8; Birk et al., J Autoimmun (1996) 9:159-66; Raz et al. Lancet (2001) 358:1749-53. The following Table is a listing of suggested antigens and their current stage of development for clinical use:

TABLE 4
Exemplary sources of antigen for clinical studies

| Antigen | Source | Current development | Reference |
|---|---|---|---|
| Proinsulin | Eli Lilly | Has been used | Revers et al., Diabetes |
| | | extensively in | (1984); 33: 762-70; |
| | | metabolic studies in | Bergenstal el al., J Clin |
| | | the 1980's | Endocrinol Metab (1984) |
| | | | 58: 973-9 |
| Insulin B9-23 peptide | Neurocrine, San | | |
| | Diego, CA, USA | | |
| Proinsulin peptide | Eli Lilly Australia | Currently in clinical | Martinez et al., J Clin |
| (without CTL epitope), | | trials | Invest (2003) 111: 1365-71 |
| intranasal use | | | |
| Ins B9-23 APL | Neurocrine, San | Phase I trials | Alleva et al., Diabetes |
| | Diego, CA, USA | completed, currently | (2002) 51: 2126-34 |
| | | in clinical trials | |
| Insulin C13-A5 | Has not been | | Arif et al., J Clin Invest |
| | produced for clinical | | (2004) 113: 451-63 |
| | use | | |
| rhGAD65 | Diamyd, Stockholm, | Phase I clinical trials | N/A |
| | Sweden | completed | |
| HSP60 (Diapep 277) | Peptor, Aventis | Phase I and II trials | Raz et al., Lancet (2001) |
| | | completed | 358: 1749-53 |

Sources of anti-CD3 mAb: The anti-CD3 mAb hOKT3γ1(Ala-Ala) is produced under GMP conditions. Other forms of anti-CD3 antibodies can also be substituted, where the anti-CD3 antibodies can be tested in preclinical experiments as described herein, in preliminary experiments as described in the preliminary data section of this Example, and in drug toxicity and safety experiments.

Drug toxicity and safety issues: Safety data has been accumulated from 32 patients with T1DM treated with hOKT3γ1(Ala-Ala) and approximately 15 other subjects treated with the drug for other conditions. Data from patients with T1DM indicate that the drug is well tolerated. In a Phase I/I trial, patients with new onset Type 1 diabetes were randomly assigned to treatment with a single course of hOKT3γ1(Ala-Ala) (n=21) or to a control (observation) group (n=20) (note that 2 of the control subjects withdrew from the trial before the 12^{th} month). The dosing regimen was modified from a 14-day to a 12-day course after treatment of the first 12 patients. The following chart shows the side effects that were seen in the 21 patients who received the 12 or 14-day dosing regimen.

TABLE 5
Side effects of treatment with hOKT3γ1 (Ala-Ala) in the
Phase I/II trial (WHO Criteria)

| Symptom/Sign | Mild | Moderate | Severe |
|---|---|---|---|
| Headache | 33% | 0 | 0 |
| Fever | 17% | 58% | 0 |
| Nausea | 8% | 0 | 0 |
| Vomiting | 8% | 0 | 0 |
| Diarrhea | 0 | 0 | 0 |
| Dyspnea | 8% | 0 | 0 |
| Myalgia | 17% | 0 | 0 |
| Arthralgia | 8% | 0 | 0 |
| Rash | 58% | 25% | 0 |
| Hypotension | 0 | 0 | 0 |

A Phase II trial of anti-CD3 mAb was initiated with a new lot of the drug. A repeat of Phase I dosing PK studies were carried out with this new lot of drug and the proposed dosing reflects the recommended dosing from these studies.

Patient population: The study population involves patients with T1DM of no longer than 6 weeks duration. This duration cutoff is arbitrary but is based on findings from the Cyclosporin trials in which the response rate was significantly better in patients who began treatment within 6 weeks compared to those that began treatment after that time. The age range is 8-30 years (Stiller et al., Science (1984) 223:1362-7). The following inclusion and exclusion criteria are proposed.

The inclusion criteria includes: 1) Diagnosis of Type I Diabetes Mellitus according to ADA criteria for no more than 2 months; 2) Males or Females ages 8-30 years of age, minimal body weight of 34 kg; 3) Detectable anti-GAD, anti-ICA512/IA-2, or insulin autoantibodies (prior to insulin treatment). Patients are not be included/excluded or randomized on the basis of HLA types. An even distribution of patients with HLA types permits the analyses proposed (if for example tetramer studies are involved) into each study group.

Sample size and justification: At the present time, there are no approved treatments for T1DM other than diet and insulin. Therefore, the study compares the combination of antigen and anti-CD3 mAb to intense glucose control with observation rather than to anti-CD3 mAb alone or antigen alone. The latter comparison is conducted with large group sizes (about 62/group). A retention of 80% of baseline C-peptide responses is selected as criteria to judge effects of treatment. Since the mean fasting C-peptide in control patients at entry was 0.21 pmol/ml, retention of even 80% of this response would have clinical significance because the stimulated level of C-peptide would be >0.2 pmol/ml, which has been associated with improved metabolic control (The DCCT Research Group, J Clin Endocrinol Metab (1987) 65:30-6). Retention of this level of C-peptide response has clear clinical significance. In the Phase I/II trial, 24% (5/21) of drug treated and 11% (2/19) control subjects met this criteria at 2 years after study entry. It is expected that the combination treatment can double this rate or that 48% of subjects should retain 80% of their baseline responses after 2 years. For this criteria, sample sizes of 23/group provide 81% power (alpha=0.05, two tailed test). This proposed sample size provides 86% power for an alpha 0.05 at 1 year.

Randomization and treatment plans: Screening laboratory studies are done after obtaining consent. If the results of these studies are satisfactory, the patient is randomized to 1 of the 4 groups. All patients undergo a 4 hour MMTT as described previously. The patients are asked to wear a continuous glucose monitor so that the mean amplitude of glycemic excursions can be compared. All of the patients in each of the treatment groups are asked to maintain a hemoglobin A1c level of <7.5% over the duration of the study. This level has been suggested by Pediatric Endocrinologists as indicative of tight glucose control without the risk of severe and frequent hypoglycemia. Modifications (increase or decrease) of this recommendation may be required by the DSMB. In order to do this, all patients are contacted by a CDE approximately every 2 weeks. This individual will contact the patient's physician and provide assistance as needed to meet the goals of the study. This effort is needed in all groups because of the evidence that glycemic control per se is an important determinant of C-peptide responses in patients with Type 1 diabetes. Because all study subjects are asked to maintain a comparable level of glycemic control, hemoglobin A1c levels will not be a primary endpoint of this study. However, the amount of insulin required to reach the desired level of glucose control will be analyzed.

Patients randomized to the combination of anti-CD3 mAb with antigen or anti-CD3 mAb alone receive a 12-day course of hOKT3γ1(Ala-Ala). The dose to be used is: Day 1: 227 μg/m²; Day 2 459 μg/m²; Days 3-12: 919 μg/m² per day. The drug is administered i.v. over 15 minutes. Patients do not need to be hospitalized for the entire 12 day treatment period but are admitted for the first 3 infusions. The patients receiving the combination commence antigen administration with the first dose of drug. Antigen is then re-administered at approximately 3-month intervals depending on the outcomes of preliminary studies and preclinical studies. Patients who are randomized to receive antigen alone can receive the first dose after the baseline studies and MMTT are completed. Patients in the anti-CD3 mAb group do not receive antigen.

Circulating lymphocyte counts, chemistries and other safety parameters, including EBV and/or CMV viral loads are monitored over the course of the 2 years. MMTT is repeated every 6 months for 2 years. At the time of screening, and throughout the 2 years of study, samples are drawn for T cellular studies described below. Based on an allowable blood draw volume of 7 cc/kg, it is estimated that the minimum body weight for this study is about 33 kg.

Analysis of endpoints: The primary endpoint is the frequency of individuals with C-peptide responses to a MMTT that are 80% of baseline levels at 2 years. The frequency of individuals that meet this criteria in the strict glycemic control and observation group and in the combination of antigen with anti-CD3 mAb group is compared by a Chi-squared analysis.

Secondary endpoints include retention of 80% of baseline C-peptide responses at 1 year. An analysis of the data, therefore, is undertaken at this time point and the frequency of individuals who meet these criteria in the strict glycemic control and observation group, and the combination of antigen with anti-CD3 mAb group is compared by Chi-squared analysis as well. Using the same approach, it is evaluated whether the frequency of individuals with 80% of baseline C-peptide responses is greater in the group receiving anti-CD3 mAb or antigen alone at 1 and 2 years, but it is not the intent to draw conclusions about differences between the 3 treatment groups. The dose of insulin that is used by individuals in the two primary groups can be compared, as well as the level of glycemic excursions as captured with a continuous glucose monitor.

Testing of whether regulatory T cells are induced or expanded is also conducted.

Rationale: In the presence of the anti-CD3 mAb, responses to islet antigens can be modulated by the present methods. Currently available technologies involving either ELISPOTs or MHC Class II tetramers, allow one to directly answer whether the combination of antigen with anti-CD3 mAb has changed the number of antigen reactive precursors as well as the phenotype of the responders. Furthermore, the preliminary studies in patients described above indicate that the mAb induces subpopulation(s) of regulatory T cells. Therefore, whether the CD4+ T cells that are induced in vivo have functional regulatory properties and whether they are specific for the antigen that is administered is determined.

Methods:

Analysis of antigen reactive T cells by ELISPOT or by tetramers: Samples from patients in the trial are analyzed by ELISPOT using techniques described by Peakman et al. or by staining with MHC Class II GAD tetramers (Arif et al., J Clin Invest (2004) 113:451-63). The ELISPOTs analyze responses to control peptides, as well as insulin peptide C13-A5 and IA-2 peptides. Both IL-10 and IFN-γ responses to proinsulin and IA-2 peptides can be studied. IL-10 responses to IA-2 appear to be the most discriminatory between control (57%) and diabetic subjects (8%), but responses to other proinsulin peptides is also desired since 72% of patients with diabetes have a IFN-γ response to at least one of either proinsulin or IA-2 peptides compared to 7% of non-diabetic control subjects.

The tetramer studies involve first an expansion of the peptide reactive cells in vitro followed by secondary stimulation of the responders with plate bound monomer followed by staining with the tetramer (Reijonen et al., Ann N Y Acad Sci (2003) 1005:82-7; Nepom et al., Arthritis Rheum (2002) 46:5-12). Supernatants are isolated from the stimulated secondary cultures for measurement of cytokines. In these studies, aliquots of cells that have been frozen can be run at the same time, and control tetramers (e.g. HA antigen) can be run at the same time as a measure of global immune suppression and/or problems with the sample after freezing.

Both techniques allow one to enumerate antigen reactive T cells and to show the relative production of cytokine in response to antigen. The treatment with anti-CD3 mAb may increase IL-10 responses to antigen, but the effects on the actual number of antigen reactive cells are not known. These responses are followed over the proposed 2 year period to determine whether repeated administration of antigen affects the responses. As described above, the phenotype of the response is expected to be maintained with continued exposure to antigen. Importantly, one will also be able to correlate these findings with changes in the metabolic course of the disease.

Studies of regulatory T cells in patients: While studies from the NOD mouse and prior studies support the notion that CD4+ cells are involved in immune regulation, the invention takes the approach that the identity of the regulatory cells is still unknown and may involve other subclasses as well. PBMC are isolated from patients at various times throughout the 2 year follow up period including time points immediately after anti-CD3 mAb treatment and after immunization with the antigen. CD4+ or CD8+ T cells are purified from PBMC using Dynal beads. Subpopulations of regulatory cells are selected for by expression of CD25. These cells are added at various ratios alone and with PBMC from the patient that have been frozen before drug treatment in the presence of PHA or antigen. The responding cells are labeled with CFSE. The antigens that are tested include the antigen that is administered to the patient with anti-CD3 mAb, as well as tetanus. Proliferation can also be stimulated with PHA. In this manner, one tests whether an inhibitory response is present and whether it is specific for the immunizing antigen or whether it is a general phenomenon. These studies can utilize frozen cells so that cells from different times can be compared. One compares the effects of CD4+ T cell addition after treatment to the same isolated from PBMC before treatment as well as comparing responses between groups. Cytokines are measured in the supernatants and in the added cells by intracellular staining. In additional cultures, anti-IL-10 and/or anti-TGF-β mAb are added to the cultures to determine if an inhibitory effect, if seen, is dependent on these cytokines. Aliquots of the added cells are studied for expression of FoxP3 by RNA expression and GITR, CTLA-4, CD25, CD62L, and CD45RO by flow cytometry, which indicates a regulatory T cell phenotype.

Subsets of cells are also purified on the basis of suggested markers of regulatory T cells including CD25, CD45RO, CD62L, and GITR. The effects of the addition of these cells to the cultures of antigen or PHA stimulated cells are tested.

In proliferative assays utilizing autoantigens, the response to antigens may be weak, with stimulation indices that are just marginally above the cutoff of 3. This may be a particular problem with the CFSE assay if the background staining is high. Therefore, to expand the proliferative response, one tests the activation of cells in response to plate-bound MHC monomers together with anti-CD28 mAb, which generally provides a much stronger stimulus. Alternatively, one can expand antigen reactive T cells from the patient before treatment and use these cells as responders and adding CD4 or CD8 cells isolated throughout the 2 year period. The effect of regulatory cells may be on the expansion of antigen reactive T cell clones or on the priming of the clones.

What is claimed:

1. A method for restoring, establishing or inducing tolerance to a self-antigen, the method comprising administering to a subject:
(a) an anti-CD3 antibody; and
(b) the self-antigen,
wherein the anti-CD3 antibody and the self-antigen are administered in an amount sufficient to restore, establish, or induce tolerance to the self-antigen in the subject.

2. A method for restoring, establishing, or inducing tolerance to a self-antigen, the method comprising:
(a) administering to a subject:
(i) an anti-CD3 antibody; and
(ii) a self-antigen;
(b) isolating T regulatory cells from the subject;
(c) incubating the T regulatory cells in vitro under growth conditions; and
(d) administering to the subject the T regulatory cells from step (c) so as to restore, establish, or induce tolerance to the self-antigen.

3. The method of claim 2, wherein step (c) comprises incubating the T regulatory cells with IL-2.

4. The method of claim 3, further comprising incubating the isolated T regulatory cells with the anti-CD3 antibody and the self-antigen.

5. The method of claim 3, further comprising incubating the T regulatory cells with antigen presenting cells (APCs) and the self-antigen.

6. The method of claim 2, wherein the T regulatory cells express CD4, CD25 and CD62L on their surface.

7. The method of claim 2, wherein the T regulatory cells express CD25, CD45TO, CD62L and GITR on their surface.

8. The method of claim 2, wherein the T regulatory cells express CD25, FoxP3, GITR, CTLA4, CD62L and CD45RO on their surface.

9. The method of claim 2, wherein the T regulatory cells express CD4, CCR4, CD62 and CD45RO on their surface.

10. The method of claim 1, wherein the self-antigen comprises a protein or a peptide fragment of the protein.

11. The method of claim 1, wherein the subject suffers from Graves disease, Hashimoto's thyroiditis, hypoglyceimia, multiple sclerosis, mixed essential cryoglobulinemia, systemic lupus erthematosus, Type I diabetes, or any combination thereof.

12. The method of claim 11, wherein the protein comprises a thyroid-stimulating hormone receptor, thryoglobulin, throid peroxidase, myelin basic protein, glutamic acid decarboxylase (GAD65), islet cell antigen 512/IA-2 (ICA512/IA-2), islet cell antigen p69 (ICA69), insulin, proinsulin, heat shock protein 60 (HSP 60), or any combination thereof.

13. A method for treating Type I diabetes, the method comprising administering to a subject:
(a) an anti-CD3 antibody; and
(b) a self-antigen,
wherein the anti-CD3 antibody and the self-antigen are administered in an amount sufficient to treat Type I diabetes or one or more symptoms associated with Type I diabetes.

14. The method of claim 13, wherein the symptom comprises reduced insulin production.

15. The method of claim 13, wherein the symptom comprises abnormal levels of blood glucose.

16. The method of claim 13, wherein the symptom comprises destruction of insulin-producing islet cells.

17. The method of claim 13, wherein the symptom comprises a mean fasting C-peptide level.

18. The method of claim 13, wherein the self-antigen comprises insulin, proinsulin, proinsulin II, insulin B9-23 peptide, a proinsulin peptide without a cytotoxic T-lymphocyte epitope, insulin C13-A5 peptide, glutamic acid decarboxylase (GAD65), ICA512/IA-2, ICA69, or heat shock protein (HSP) 60.

19. The method of claim 1, wherein the anti-CD3 antibody and the antigen are initially administered on the same day.

20. The method of claims 1, wherein the anti-CD3 antibody is a monoclonal antibody.

21. The method of claim 20, wherein the antibody comprises an IgG molecule.

22. The method of claim 20, wherein the antibody comprises a humanized antibody or a fully human antibody.

23. The method of claim 20, wherein the antibody comprises at least two antigen binding sites.

24. The method of claim 20, wherein the anti-CD3 antibody comprises an antibody fragment.

25. The method of claim 24, wherein the antibody fragment comprises a (Fab′)₂ molecule.

26. The method of claim 24, wherein the antibody fragment does not bind to an Fc Receptor.

27. The method of claim 20, wherein the anti-CD3 antibody comprises a non-mitogenic antibody.

28. The method of claim 1, wherein the anti-CD3 antibody comprises an OKT3 antibody.

29. The method of claim 28, wherein the OKT3 antibody is a human OKT3γ (Ala-Ala) antibody.

30. The method of claim 1, wherein the administration comprises administration of an expression vector that encodes the self-antigen.

31. The method of claim 1, wherein the anti-CD3 antibody is administered intravenously.

32. The method of claim 1, wherein the self-antigen is administered intranasally, orally, subcutaneously, intramuscularly, or intravenously.

33. The method of claim 1, wherein the anti-CD3 antibody and the self-antigen is administered with a pharmaceutically acceptable carrier, excipient or diluent.

34. A kit comprising:
(a) an anti-CD3 antibody;
(b) a self-antigen; and
(c) instructions for coadministration of the anti-CD3 antibody and the self-antigen.

35. The kit of claim 34, wherein the instructions comprise a dosing schedule and dosing amounts for the anti-CD3 antibody and the self-antigen.
